(19) United States
(12) Patent Application Publication
(10) Pub. No.: US 2004/0024006 A1
(43) Pub. Date: Feb. 5, 2004

(54) Opioid pharmaceutical compositions

(76) Inventor: David Lew Simon, Mansfield Center, CT (US)

Correspondence Address: David L. Simon, P.O. Box 618, 100 Cemetery Road, Mansfield Center, CT, 06250, US

(21) Appl. No.: 10/628,089

(22) Filed: Jul. 25, 2003

Related U.S. Application Data
(63) Continuation-in-part of application No. 10/306,657, filed on Nov. 27, 2002.
Application No. 10/306,657 is a continuation-in-part of application No. 09/922,873, filed on Aug. 6, 2001, now Pat. No. 6,569,866.
Application No. 09/922,873 is a continuation-in-part of application No. 09/152,834, filed on Sep. 14, 1998, now Pat. No. 6,271,240.
Application No. 09/152,834 is a continuation-in-part of application No. 08/866,334, filed on May 30, 1997, now abandoned.
Application No. 09/152,834 is a continuation-in-part of application No. 08/643,775, filed on May 6, 1996, now abandoned.

(51) Int. Cl. A61K 31/485

(52) U.S. Cl. 514/282

(57) ABSTRACT

The invention is directed in part to dosage forms comprising a combination of an analgesically effective amount of an opioid agonist analgesic and a neutral receptor binding agent or a partial mu-opioid agonist, the neutral receptor binding agent or partial mu-opioid agonist being included in a ratio to the opioid agonist analgesic to provide a combination product which is analgesically effective when the combination is administered as prescribed, but which is less analgesically effective or less rewarding when administered in excess of prescription. Preferably, the combination product affects an opioid dependent individual differently from an opioid naïve individual, and has a diminished likelihood of being associated with a life-threatening adverse drug reaction, especially in the opioid dependent individual.

### RELATED APPLICATIONS

This patent application is a continuation-in-part of co-pending U.S. patent application Ser. No. 10/306,657 filed Nov. 27, 2002, which is a continuation-in-part of U.S. patent application Ser. No. 09/922,873, filed Aug. 6, 2001, now U.S. Pat. No. 6,569,866, which is a continuation application of U.S. patent application Ser. No. 09/152,834, filed Sep. 14, 1998, now U.S. Pat. No. 6,271,240, which is a continuation-in-part of U.S. patent application Ser. No. 08/866,334, filed May 30, 1997, abandoned on May 12, 1998, and U.S. patent application Ser. No. 08/643,775, filed May 6, 1996, abandoned on Sep. 22, 1998, the disclosure of each which is hereby incorporated by reference.

### FIELD OF INVENTION

The present invention relates to novel pharmaceutical compositions for improved administration of drugs of the opioid classification to a human or animal.

### BACKGROUND OF THE INVENTION

An opioid agonist analgesic is a drug or pharmaceutical agent that traditionally is used to treat pain, to suppress coughing, to treat diarrhea, and for other medicinal uses. Depending upon the degree with which a particular opioid agonist medication binds to specific opioid receptor subtypes, such as its affinity for one opioid subtype receptor in preference to another, the opioid agonist analgesic may tend to cause euphoria, or it may tend to cause dysphoria. Some opioid analgesic agonists may also tend to cause nausea by stimulating or inhibiting areas in the brain known as “the vomiting center” and “the chemotactic zone,” depending upon the degree with which specific opioid receptor subtypes are activated, and depending to some extent upon the ability of a particular opioid agonist analgesic to penetrate the blood-brain-barrier (BBB). Examples of opioid receptor subtypes are delta-receptors, kappa-receptors, mu-receptors and sigma receptors. These opioid receptor subtypes may be further subcategorized, as for example, mu₁-receptors and mu₂-receptors.

The opioid antagonist nalmefene has unique characteristics which set it apart from other opioid antagonists such as, for example, naloxone and naltrexone. The unique opioid receptor subtype binding profile of nalmefene enables nalmefene alone, as compared to naloxone and naltrexone, to allow preferred antagonism of opioids at the kappa-opioid receptors versus the mu-opioid receptors, which in turn results in an optimal homeostatic balance of dopamine.

Szekely shows a schematic representation of two opposing opioid systems located in the mesolimbic system of the human central nervous system. These systems modulate A10 dopaminergic neurons projecting in the nucleus accumbens. As illustrated in this reference, stimulation of mu-opioid receptors (the mu subtype of opioid receptor) in the ventral tegmental area (VTA), the site of origin of the A10 neurons, increases dopamine release in the nucleus accumbens (NA). Selective blockade of this mu-receptor results in significant decrease in dopamine release in the nucleus accumbens. In stark contrast, stimulation of kappa-receptors (the kappa subtype of opioid receptor) in either the VTA or the NA results in a decrease in the amount of dopamine released. Selective blockade of kappa-receptors significantly increases dopamine release.

Spanagel et al. demonstrate that tonically active and functionally opposing mu and kappa opioid systems regulate mesolimbic dopamine release in the nucleus accumbens. They report that the injection of mu-opioid agonists such as DAGO into the VTA stimulate mu-opioid receptors and increase the release of dopamine from the VTA into the NA. As would be expected, administration of a mu-opioid receptor antagonist into the VTA decreases dopamine release. The authors further report that kappa-opioid receptors agonists such as U-6953 infused into the NA inhibit dopamine release there, whereas kappa-opioid receptor antagonists such as nor-BNI increase dopamine release. An “agonist” is a “like” chemical with similar action to a given drug. An “antagonist” is a chemical, often with a similar chemical structure to a given drug, which exerts a dissimilar action to the given drug, in general preventing the “like” action of that given drug. With opioid receptors, in general, an agonist binds to the receptor and activates it in such a way as to begin a cascade of chemical or pharmacological events so as to result in the end effect related to a particular opioid receptor subtype. In contradistinction, an antagonist will bind to the receptor but not activate it. An antagonist exerts its actions by blocking the receptors from agonists, by physically occupying the space on the receptor where an agonist would otherwise bind.

The opposing mu and kappa opioid systems acting together provide a homeostasis of dopamine levels within the central nervous system. Changes in these opioid systems, such as by activation or blockade of the specific receptors, would therefore be expected to modulate opioid-induced effects that are mediated by mesolimbic pathways. Mu and kappa receptors are found elsewhere in the human body. For example, they have been located in the spinal cord (See Fujimoto, Bakshi and Behrmann, below) and in other non-central nervous system organs such as the kidney and intestine (See Ohnishi and Kreek, below). Accordingly, the model presented provides a neurochemical framework for understanding the adaptive changes resulting from long term use of opioids, as well as the clinical response elicited by exogenously administered opioid agonists and antagonists having different binding profiles.

For example, Pan et al report modifications in opioid-induced behavior resulting from changes in these mu and kappa systems. These authors state that the effects of opposing mu and kappa receptors extend to opioid action on emotion, perception and drug reinforcement. While morphine and other mu-opioid agonists increase dopamine release and produce euphoria and place preference, kappa-opioid agonists reduce mesolimbic dopamine release and produce dysphoria and aversion.

Scientists have shown that nalmefene, relative to other opioid antagonists such as naloxone and naltrexone, is significantly more kappa-receptor preferring. By way of example, Kreek et al. conclude that nalmefene has more kappa binding activity than either naloxone or naltrexone. Specifically, nalmefene is more potent than either naloxone or naltrexone as a kappa-receptor antagonist, and therefore would block kappa agonists (e.g. the naturally occurring dynorphin) to a greater extent than the other antagonists.

Fujimoto et al. demonstrate differences between mu and kappa receptor effects in the spinal cord. Specifically, these authors report that the administration of dynorphin, a potent kappa agonist, results in decreased analgesia. The dynorphin causes antianalgesic effects at the level of the spinal cord. Fujimoto shows that when a kappa-opioid receptor antagonist such as Cholera Toxin is given, the antianalgesic effect of dynorphin is inhibited.

Bakshi et al. shows that kappa receptors are widely distributed in the spinal cord, and that administration of dynorphin causes motor impairment. These authors also demonstrate that nalmefene is selective for these intraspinal kappa receptors, and limits dynorphin induced motor dysfunction after spinal cord injury.

Behrmann et al. report that a single dose of nalmefene has increased activity at kappa receptors and that a single dose of nalmefene exerts a significant neuroprotective effect after acute spinal cord injury, in direct contrast to the mu-preferring opioid antagonist naloxone that showed no significant effect on neurological recovery after spinal cord injury.

Ohnishi et al. teach the effects on urine production due to kappa-opioid receptor pharmacology at both the level of the pituitary gland and the kidney.

Crain et al. (U.S. Pat. No. 5,580,876) teach a method for “selectively enhancing the analgesic potency of a bimodally-acting opioid agonist” which shows that nalmefene, much more so than other opioid antagonists, enhances analgesia produced by opioid agonist analgesics. Crain et al. further teach that much lower concentrations of nalmefene are required to enhance analgesia than with either naloxone or naltrexone, thus further supporting that nalmefene optimizes dopamine homeostasis to a much greater extent than other opioid antagonists such as naloxone and naltrexone.

The prior art contains many examples of methods for prolonged delivery of naltrexone. Naltrexone implants, depots and other sustained release formulations of naltrexone have be described in great detail. These naltrexone preparations have been proposed as improved methodologies for treating addiction to opioid agonist analgesics. What has not been appreciated in the prior art are the unique pharacological and clinical advantages provided by the prolonged administration of nalmefene via sustained delivery formulations such as sustained release formulations for per os administration, subcutaneous implants, injected depot preparations for subcutaneous or intramuscular administration and transdermal delivery systems.

A significant problem in treating humans addicted to opioid agonist analgesics with per os naltrexone is the significant gastrointestinal upset which is often caused soon after per os administration of this drug. Thus, to encourage use of opioid antagonists for addiction treatment, it is important to formulate a delivery system of opioid antagonist that is administered in other than per os form. Such a delivery system would tend not to dissuade a human from being administered an opioid antagonist, even if it were not in a sustained delivery formulation. Examples of such delivery routes are buccal, intranasal, sublingual, transdermal and transmucosal preparations, including suppositories for rectal administration. These routes of delivery, even if not delivered over a very prolonged time, still would increase patient compliance with opioid antagonist administration by allowing a third party to administer, or to observe self-administration, of the opioid antagonist. For example, a “squirt” through the nares and onto the nasal mucosa would ensure a delivered dose of antagonist Further, by bypassing the gastrointestinal tract, such intranasal administration is much less likely to cause gastrointestinal upset. Intranasal administration has the further advantage, as does sublingual administration, of bypassing metabolism by the liver upon initial administration. Metabolism of a drug by the liver after delivery to the gastrointestinal tract is generally referred to as “first pass metabolism,” and is a significant disadvantage for per os administration of many drugs. Nalmefene and naltrexone are two drugs that undergo very significant first pass metabolism. Of these two drugs, nalmefene is very much preferred for the treatment of opioid addiction because of its unique opioid receptor subtype binding profile compared to naltrexone, as described above.

The administration of opioid antagonists cause upregulation of opioid receptors present on the surface of cell of the central nervous system. The result of this increased density of opioid receptors is that more opioid receptors will then be available to the naturally occurring endogenous endorphins that are in proximity to these receptors. Because beta-endorphin production is decreased by a mechanism generally known as “negative feedback inhibition” in humans who are chemically dependent upon, and who are still being administered, exogenous opioid agonist analgesics, immediately upon cessation of opioid agonist analgesic administration there is a lack of beta-endorphin in these humans relative to the normal state in humans not chemically dependent upon opioid agonist analgesics. Thus, administration of opioid antagonists not only increase the number of receptors for beta-endorphin to bind to, in addition, these antagonists actually stimulate the production of endorphins by causing the release of negative feedback inhibition of its production. Thus, the cellular changes induced from chronic use of opioid agonist analgesics are reversed to a significant extent. Beta-endorphin attaches to and activates mu-opioid receptors, which results in a cascade of biochemical reactions, the result of which is an increase in central nervous system (CNS) dopamine. These changes brought upon by treatment with an opioid antagonist, such as nalmefene, restore to a human being a more normal physiological state, which will decrease the human's cravings for, and reduce the human's tolerance to, exogenously administered opioid agonist analgesics.

This upregulating effect of opioid antagonists in humans for treating addiction to opioid agonist analgesics has not been appreciated by those skilled in the art, particularly in the case of nalmefene which provides distinct pharacological and clinical advantages over other opioid antagonist for treating addiction to opioid agonist analgesics. Nalmefene tends to optimize CNS dopamine by virtue of its greater affinity for kappa-opioid receptors relative to mu-opioid receptors, as compared to naltrexone and other opioid antagonists.

A sufficiently high concentration of opioid antagonist must be present at the opioid receptor blocked, e.g. at a mu₁-opioid receptor, to prevent an exogenously administered opioid agonist analgesic or its metabolite from binding to the receptoror, but not such a high concentration as to totally block binding of endogenous beta-endorphin to that receptor. Again, nalmefene is the unique opioid antagonist which will block beta-endorphin at mu₁-opioid receptors to a relatively lesser extent than other antagonists such as naloxone and naltrexone, while at the same time having optimal blocking of kappa-opioid receptors by endogenous molecules such a dynorphins. Therefore, nalmefene alone, as compared to naloxone and naltrexone, not only optimizes dopamine regulation during detoxification, but also following detoxification. Thus, nalmefene is not an analogous compound to other opioid antagonists because nalmefene provides distinct pharmocological and clinical advantages for post detoxification treament of patients addicted to opioid narcotics not available with other opioid antagonists.

### SUMMARY OF THE INVENTION

The present invention comprises methods of administering the medicinal agent nalmefene, with or without co-administration of a centrally-acting dopaminergic drug such as bupropion. In one aspect, the invention provides a method for administering nalmefene which acts to produce a prescribed serum concentration of nalmefene over some time period that optimally regulates dopamine release in the central nervous system. In a second aspect, the invention provides a method for administering nalmefene which bypasses the gastrointestinal tract and therefore eliminates “first pass” liver metabolism and also avoids gastrointestinal discomfort. In a third aspect, the invention provides a method of administering nalmefene which results in a relatively gradual release of nalmefene over time when administered enterally so as to avoid large peaks in serum nalmefene concentration after per os administration.

The pharmacological and clinical advantages provided by these methods can only be achieved using the opioid antagonist nalmefene. As discussed above, nalmefene alone, in stark distinction from other opioid antagonists such as naloxone and naltrexone, has unique binding affinities for opioid-receptor subtypes, namely mu-receptors and kappa-receptors. The unique binding profile of nalmefene allows for preferred blocking at kappa-receptors relative to mu-receptors, such that dopamine release will tend to be less inhibited due to actions at kappa-receptors than would be the case with equivalent blocking at mu-receptors by other opioid antagonists such as naloxone or naltrexone.

### DETAILED DESCRIPTION OF THE INVENTION

Humans addicted to opioid agonist analgesics, such as buprenorphine, codeine, fentanyl, heroin, meperidine, methadone, morphine, opium, oxycodone, sufentanyl, and many other drugs classified as opioid narcotics, have a very difficult time abstaining from self-administering these analgesics, especially after detoxification and during the process associated with detoxification that is generally known as “withdrawal.” The present invention fulfills a long-awaited need to aid such humans so that addiction treatment for chemical dependencies on opioid agonist analgesics is greatly enhanced.

The invention encompasses a variety of methods for administering nalmefene that produce relatively constant release of nalmefene into the bloodstream of a human for a relatively prolonged or sustainable time. Thus, the serum concentration of nalmefene is less likely to have significant peaks and troughs over time as seen in association with intravenous bolus injections of nalmefene, or per os administration of nalmefene in a non-sustained release form.

The invention further provides for a practical way of accomplishing the above stated ends. For example, nalmefene can, and has been, administered by constant intravenous infusion in a post-surgical setting or following opioid overdose. However, this method has not been used as a method for addiction treatment. Further, intravenous infusion is cumbersome and not at all practical in ambulatory humans, especially those prone to opioid agonist analgesic addiction.

By stark contrast to a constant intravenous infusion of nalmefene, the present invention allows for nalmefene to be constantly absorbed into the bloodstream by way of very small capillaries found within living human tissue at prescribed constant rates, such as by diffusion through skin with transdermal delivery, by diffusion through fat with subcutaneous delivery—either by surgical implantation or needle injection into fatty tissue, by gradual absorption through the gastrointestinal tract in a sustained-release per os delivery method, by absorption through muscular tissue as with intramuscular injection, by absorption through mucosa as found in the gastrointestinal tract, or by diffusion through mucosal membranes as found in the sublingual area of the mouth or in nasal passages.

The following examples illustrate the present invention:

### EXAMPLE 1

There are a variety of transdermal delivery systems known in the art which deliver an array of medicinal agents in a sustained and constant fashion. Examples are Androderm® and Testoderm®(systems that deliver testosterone, Alora™, Climara®, Estraderm® and Vivelle® systems which deliver estradiol, Catapres-TTS systems that deliver clonidine, Duragesic® systems which deliver fentanyl, Deponit®, Nitro Dur® and Transderm-Nitro® systems that deliver nitroglycerin, Habitrol®, Nicotrol® and ProStep® systems which deliver nicotine, and Transderm Scop® that delivers scopolamine.

The ideal steady state plasma concentration of nalmefene for blocking the effects of exogenously administered opioid agonist analgesics at mu-opioid receptors, while simultaneously allowing beta-endorphin to bind to and activate mu-opioid receptors, and effectively blocking dynorphins at kappa-receptors, in humans addicted to opioid agonist analgesics, is from about 1 to about 3.7 ng/ml, most preferably between about 1.25 and about 2.5 ng/ml, such as 2.15 ng/ml. For a 70-kilogram (kg) adult (but not elderly) human, a sustained steady state plasma concentration for nalmefene of 2.0 ng/ml can be achieved by a transdermal delivery system in the following way.

Assuming an elimination constant (a.k.a Kₑ) for nalmefene of 0.0642 hr⁻¹ (which is a value for Kₑ that is furnished by a distributor of nalmefene), and assuming an average volume of distribution (a.k.a. V_{d}) for nalmefene of 8.6 liters/kg (which has been shown to be an approximate average V_{d} in non-elderly adult humans for nalmefene), a target serum concentration in a 70 kg adult of 2.0 ng/ml nalmefene can be maintained by administering parenteral nalmefene, as administered transdermally, at an input rate of approximately 1.8 to 2.0 mg per day.

2.0 mg/day nalmefene can be effectively administered transdermally by constructing a transdermal delivery system described as follows: FIG. 1 illustrates a transdermal delivery system as taught by the present invention. The system is embodied in a transdermal patch, generally designated 10, comprising drug reservoir 12 which includes a matrix 14 having nalmefene base and PEGML dispersed therethrough. The reservoir 12 is covered by a impermeable backing layer 16 which is sized slightly larger in circumference than the reservoir. An adhesive overlay 18 is provided for adhering the patch to the surface of the patient's skin. The overlay is separated from the reservoir 12 by the peripheral portion 20 of the backing layer 16 surrounding the reservoir 12. This is required to prevent adverse reactions between the PEGML dispersed in the reservoir and the adhesive supported on the overlay. The patch 10 further includes an adhesive release liner 22 which is removed by the patient or clinician just prior to attaching the patch to the skin.

A number of different materials are suitable for forming the matrix 14. However, due to the solubility characteristics of PEGML, the matrix is preferably formed from an anhydrous material such as natural or synthetic rubbers other polymeric materials, thickened mineral oil or petroleum jelly, when PEGML is used as the flux enhancing compound. In the illustrated embodiment, the matrix is formed from an ethylene vinylacetate copolymer preferably having an vinylacetate content of from about 28% to about 60%.

The nalmefene is dispersed through the matrix at a concentration in excess of saturation, with the amount in excess of saturation being determined based on the intended useful life of the patch. Accordingly, the typical concentration of nalmefene in the reservoir is in the range of from about 10% to about 35% by weight. The PEGML is dispersed through the matrix at a concentrations below saturation and preferably between the range of activity of from about 0.25 to about 0.60. Thus, the reservoir typically contains from about 25% to about 60% PEGML by weight. Where various PEGML compositions having different average molecular weights of the PEG component can be utilized, a composition comprising PEG (200-400) ML is preferred.

FIG. 2 illustrates a second embodiment of the transdermal patch. As shown in FIG. 2, the patch, generally designated 100, comprises a laminated reservoir 110 including layers 112 and 114. Layer 112 comprises a drug/flux enhancer reservoir substantially as described with respect to the reservoir 12 of FIG. 1. Thus, layer 112 includes as ethylene-vinyl acetate matrix 116 having nalmefene and PEGML dispersed therethrough. Layer 114 includes a PEGML reservoir that utilizes essentially the same matrix material 118 as that found in layer 112. The matrix 118 has PEGML dispersed therethrough but is substantially free of any undissolved nalmefene.

The patch 110 further includes a semi-permeable membrane 120 between layers 112 and 114 which controls the release of PEGML from layer 114 into layer 112 and from layer 112 into the skin. The membrane 120 may be formed from any pharmaceutically acceptable material having low permeability to PEGML, and in the preferred embodiment the membrane is formed from ethylene-vinyl acetate copolymer having a lower vinyl acetate content than the matrix.

The advantage of the FIG. 2 embodiment is that the nalmefene is concentrated in layer 112 near the surface of the skin, rather than throughout the entire reservoir as is the case with reservoir 12 in FIG. 1. This permits reduced loading of nalmefene in the patch, while at the same time providing for a sufficient PEGML reservoir for the intended life of the delivery system.

The patch 100 further includes an impermeable backing layer 122 superimposed over the reservoir 110 and an adhesive overlay 124 as described above with respect to the FIG. 1 embodiment. Also included is a release liner 126 that is removed just prior to attaching the patch to the skin.

To provide the desired plasma concentration of 2.0 ug/ml as described above, the patch is constructed, in one example, according to the FIG. 1 embodiment. The matrix is formulated with weight percentages of nalmefene and PEGML to provide an input rate of nalmefene of 20 ug/cm²/hr. A patch having this input rate is dimensioned to present approximately 4.2 cm² of reservoir surface area in contact with the skin. Typically, the patch is configured to be substantially square-shaped, though it may be round, oval or of another shape having a similar area, and in a square formation measures approximately 2.05 cm×2.05 cm. In another example, the patch is formulated to deliver an input rate of 3.5 ug/cm² of nalmefene per hour. To provide the required plasma level, the size of the patch must be substantially larger. A patch of this type is dimensioned to present 23.8 cm² of reservoir surface area in contact with the skin. A circular embodiment of this patch is configured as a circle having a radius of approximately 2.8 cm, and it is significantly larger than the previously described example. With either of these examples, steady state nalmefene concentrations can be reached faster by giving a loading dose of nalmefene, e.g. by intravenous bolus as in rapid opioid detoxification under anesthesia. Thus, the present invention is an extension of U.S. Pat. No. 5,783,583, which describes loading a human with nalmefene under anesthesia, then following the loading dose with a constant delivery of nalmefene.

To maintain a steady state plasma concentration of nalmefene of 2.15 ng/ml in a 80 kg human being, assuming a Vd of 8.6 L/kg and a Ke of 0.0642 hr⁻¹, a patch delivering an amount of nalmefene of approximately 2.3 mg per 24 hour period can be formulated as having an area of about 9.6 cm² if the percent by weight of nalmefene and flux enhancing compound is formulated to yield an input rate of nalmefene of about 10 ug/cm²/hr. An embodiment of such a patch could be a circular patch with a diameter of about 3.5 cm.

As the foregoing demonstrates, the size and input rate of a prescribed series of transdermal patches can be individually altered to provide transdermal dosages of nalmefene consistent with the present invention. Alterations in flux enhancers and other materials making up the transdermal patch are likewise applicable to the present invention. Thus, upregulation of opioid receptors, stimulation of endogenous beta-endorphin release, and optimal blocking effects at both mu- and kappa-opioid receptors by nalmefene, which all serve to optimally regulate dopamine release, can be accomplished without it being necessary for the patient to return to the clinic daily over the extended term of a nalmefene maintenance program.

Additional advantages result from the continuous and sustained nature of transdermal delivery of nalmefene. Because the drug becomes absorbed into the dermis, removing a transdermal patch does not instantaneously stop drug administration. The lag between the time the patch is removed and the time the drug ceases to be absorbed into the bloodstream is an effective tool against the compulsive behavior that is typical of opioid addicted humans who seek immediate gratification from their actions. If an addicted human wanted to stop nalmefene delivery in order to experience the effects of exogenous opioid agonist analgesics, this would have to be planned out in advance by removing the transdermal delivery system some time ahead of the anticipated drug use. Thus, impulsive actions on the part of the addict would not result in immediate results. Such a lapse, in many instances, is sufficient to deter the addicted human from impulsively discontinuing nalmefene therapy. In addition, the removal of the patch by the patient is quite apparent to the support person or clinician monitoring the patient, thus making the process of monitoring easier and more effective.

It may be the case that the dosage schedule over the course of a nalmefene maintenance protocol will have to be tailored for each individual patient. The transdermal delivery system of the present invention is ideally suited for individualized dosage regimens since the size, delivery rate and number of patches can be readily designed to meet the needs of a particular patient. Variations among patients include mass (weight in kilograms), volumes of distribution, and the particular state of opioid receptor regulation at a given time.

The transdermal delivery of nalmefene in an appropriate dosage negates the effects of exogenously administered opioid agonist analgesics while maintaining the effects of the natural opioid endorphin system to the greatest degree possible. The constant delivery of nalmefene results in relatively constant serum concentrations, so as not to result in high peaks of nalmefene concentration as occurs following a bolus administration of the drug. This is especially important in treating addiction to opioid agonist analgesics, because if a high peak concentration of nalmefene is reached after each bolus, the concentration of nalmefene at mu-opioid receptors may become high enough to block not only exogenous opioid agonist analgesics, but naturally occurring endorphins as well. This would be expected to result in dysphoria or other unpleasant effects. Such unpleasant effects, if repeatedly associated with being administered nalmefene, may result in the development of an aversion to being administered the drug. This dissuades the human from being compliant with a prescribed regimen of nalmefene administration. Humans addicted to opioid agonist analgesics are notoriously unreliable in following a regimen of self-administer medications per os. Thus, transdermal delivery of an antogonist can provide important advantages with respect ot patient compliance, since addicted humans will exhibit a much higher compliance rate for the full term of the nalmefene maintenance protocol.

While it may be necessary for patients to periodically replace a number of transdermal patches to complete a nalmefene maintenance protocol, this can be accomplished under the supervision of a support person designated to assist in, and to monitor, the treatment of the addicted human. Monitoring can be facilitated by placing the transdermal patch on the surface of the patient's skin and marking the edge of the impermeable backing layer and a corresponding portion of the skin surface in one or more locations with indelible marker. Marking the patch and the skin in this manner registers the patch with the skin, such that if the addicted patient removed the patch it would be difficult for him or her to replace it with the patch in the exact orientation prior to removal. Using this method, addicts can easily be monitored since the support person or a clinician can readily determine if the patch had been left in place.

### EXAMPLE 2

Another way of delivering nalmefene both enterally in small incremental doses (as with normal swallowing) and parenterally (due to absorption sublingually) over a relatively prolonged period is to formulate a chewing gum preparation, such as nalmefene polacrilex, in a fashion somewhat resembling nicotine polacrilex which is marketed as Nicorette gum by SmithKline Beecham.

By formulating a nalmefene polacrilex gum with a given mass-unit of nalmefene per individual unit of chewing gum, the prescribed number of units of chewing gum can be administered to a human per over a prescribed amount of time to yield the preferred serum concentration of nalmefene, the prescribed number of units of gum per time-unit depending upon the lean body mass of a particular human.

### EXAMPLE 3

Sustained administration of nalmefene may also be accomplished by surgically implanting an osmotic pump. The Alza Corporation manufactures osmotic pumps; one example is the surgically implantable ALZET® Osmotic pump, and another example is the OSMET osmotic pump for rectal administration. Both are capable of delivering nalmefene within the scope of the present invention.

For example, if the desired parenteral input rate into a human is 2.4 mg/day, then using ALZET® Osmotic Pump model #2ML1 that delivers liquid at a rate of 10 microliters (ul) per hour, or 240 ul/day, if the concentration of nalmefene is 10 mg/milliliter (10 mg/cc), the desired input rate can be achieved. To thwart local tissue immunological reactions and pump “encapsulation,” a small dose of triamcinolone may be included in the osmotic pump for release to local tissue surrounding the implanted pump. In order to avoid subjective discomfort due to this foreign object being implanted subcutaneously, a pharmacologically compatible local anesthetic may also be included within the pump.

The particular osmotic pump embodied herein is described for illustrative purposes only, and is not intended to limit the scope of the present invention, which is consistent other osmotic pump release devices.

### EXAMPLE 4

Nalmefene may be prepared as nalmefene polistirex, in a fashion similar to the known preparation of dextromethorphan polistirex. Such a form of dextromethorphan polistirex is manufactured by Mediva Pharmaceuticals, Inc. in Fort Worth, Tex. and is marketed as Delsym®, which provides an extended release of dextromethorphan over approximately 12 hours.

The preferred dose of nalmefene polistirex is based on the lean body mass of the treated human. By formulating an elixer of nalmefene polistirex with a given mass-unit of nalmefene per volume-unit, the prescribed amount of nalmefene can be administered that results in the preferred serum concentration of nalmefene.

### EXAMPLE 5

There are a variety of intranasal delivery systems in the prior art that deliver various medicinal agents in a parenteral, non-intravenous fashion via absorption through the nasal mucosa. Examples are Atrovent® nasal spray that delivers ipatropium bromide, Flonase® nasal spray which delivers fluticasaone propionate, Stadol NS® which delivers butorphanol tartrate, Beconase AQ® nasal spray that delivers beclomethasone dipropionate monohydrate, Nicotrol® NS nasal spray which delivers nicotine, Miacalcin® nasal spray which delivers calcitonin-salmon, DDAVP® nasal spray which delivers desmopressin acetate, Nasacort® AQ nasal spray and Nasacort® nasal inhaler which deliver triamcinolone acetonide, Nascobal™ gel that delivers cyanocobalamin, and Astelin® nasal spray which delivers azelastine hydrochloride.

According to the present invention, nalmefene is prepared as a free base or in its salt form and incorporated into a pharmacologically suitable nasal carrier, in a manner known to those skilled in the art. The choice of suitable carrier will depend upon whether the route of administration is by nasal solution, nasal suspension, or nasal aerosol using a volatile propellant. Generally, water is used in formulating a preparation, and the pH of the preparation may be altered by any one of known pH adjusters, e.g. sodium hydroxide.

A tartrate, stearate or palmitate formulation of nalmefene may be used, or nalmefene may be in the form of nalmefene hydrochloride, and formulated such that 1 gram of active nalmefene is mixed with 80 ml of distilled water, then adjusted to a pH of approximately 7.4 with dilute sodium hydroxide, and then isotonic saline is added along with a suitable preservative and antibacterial agent, to yield a total volume of 100 ml. This yields a nalmefene solution with a concentration of 10 mg/ml nalmefene. This final solution is passed through a 0.2 micron Millipor filter to remove bacteria and other undesired particles. The filtered solution is then placed aseptically into a container to which is then attached a metered dosing mechanism which allows approximately 0.1 ml to be delivered in each spray. An example of such a metered dosing system in found with the commercially marketed Stadol NS® (Bristol-Myers Squibb Co.). One spray to one nostril may be expected to yield a blood serum concentration of approximately 1 ng/ml 30-90 minutes after administration. Because the serum half life of nalmefene is approximately 10.8 hours, 5 consecutive 1 mg doses approximately 11 hours apart will result in steady state serum concentrations of nalmefene of approximately 1 ng/ml. Alternatively, the human may be given a loading dose of intravenous nalmefene and then transferred to a nasal administration regimen. A practical dosing schedule for nalmefene may be 1.5 mg intranasally every 12 hours. To facilitate this dosing regimen, 1.5 g of nalmefene may be substituted for the 1.0 mg previously described for preparation of a nasal solution, thus yielding a final concentration of 15 mg/ml, which can be administered in 0.1 ml increments.

In addition, permeation enhancers may be added to the nasal solution to increase input through the nasal mucosa Palmitoyl and stearyl components of lysophosphatidylcholine in 0.5% concentration, are examples of mucosal permeation enhancers. Lysolecithin is a very potent mucosal permeation enhancer.

It is understood that these examples are for illustrative purposes only and are not to be construed as limiting the invention in spirit or scope.

### EXAMPLE 6

Propellant-based aerosol systems for immediate non-intravenous parenteral delivery of a medicinal agent through oral mucosa sublingually are known in the art. For example, Nitrolingual® spray delivers nitroglycerin, which circumvents first-pass liver metabolism. This formulation utilizes dichlorodifluoromethane and dichlorotetrafluoroethan as propellants. Like propellants can be used in an aerosolized formulation of nalmefene which would be concentrated to deliver a dosing regimen similar to that described in Example 5. The manner of preparing a suitable formulation would be apparent to one skilled in the art in light of the present invention.

### EXAMPLE 7

There are a variety of depot preparations for subcutaneous or intramuscular injection which provide for sustainable delivery of medicinal agents at a relatively even rate. These may employ particular methods that vary from one depot system to another. Examples include Lupron Depot® systems that deliver leuprolide acetate, Depo-Provera® that delivers medroxyprogesterone acetate and Zoladex® which delivers goserelin acetate.

The method for a sustainable releasing formulation of nalmefene, which is easily parenterally injected into subcutaneous or muscular tissue, may be as simple as preparing nalmefene in an oil base such as sterile peanut oil. More elaborate systems allow for a more controlled rate of release such that steady state serum concentrations of nalmefene are more constant. These systems may entail putting microencapsulated particles of nalmefene into a suspension that can then be delivered through a percutaneous needle. For instance, a polymer of a natural compound or compounds, such as a polymer or copolymer which includes biodegradable poly-lactic and poly-glycolic acids, polylactic acid, polyglucolic acid, polylactones, or any of a number of biodegradable non-toxic polymers, is used to encase or encapsulate particles of nalmefene. Poly(L(+)-lactic acid) and DL-lactic acid have been used in the prior art for sustained release drug formulations. These “microcapsules” are then suspended in a carrier solution. After being injected parenterally, preferably by subcutaneous route, the microcapsules break down over time thereby releasing active nalmefene for capillary absorption into the bloodstream. By varying the ratio of polymers in a copolymer, or by using different polymers or coploymers in a given suspension and by varying the size of the microcapsules, the nalmefene can be released “in waves” from the suspension. In light of the present invention, one skilled in the art would be able to formulate a particle size of nalmefene, a microcapsule of the required size and composition, such that nalmefene would be released in a sustainable fashion while yielding relatively constant steady state serum concentrations of nalmefene consistent with the present invention.

As noted above, inclusion of a steroidal anti-inflammatory agent, e.g. triamcinolone, and a pharmacologically compatible local anesthetic, may provide the added benefits of greater comfort to the human administered the composition, as well as providing a means to decrease local tissue inflammatory responses which may cause induration or pain at the injection site.

### EXAMPLE 8

There exists in the prior art a variety of surgically implantable delivery systems, one such example is the Norplant® system that delivers levonorgestrel. Grossman et al in U.S. Pat. No. 5,633,000 ('000) teach a subcutaneous implant comprising a poly(ethylene-vinyl acetate) matrix in which active drug is embedded. Nalmefene is not an equivalent drug to the active drug in claimed in '000. Further, the preparation of '000 is alleged to be “non-inflammatory, biocompatible and non-biodegradable,” which therefore results in a prolonged, controlled release of active drug with “near zero-order” kinetics.

### EXAMPLE 9

The prior art shows many sustained- or controlled-release tablets and capsules for per os administration. The oral controlled-release system is often made of polymers that release active drug by diffusion, bio-erosion, or swelling due to increased osmotic pressure generated in the gastrointestinal tract. Diffusion controlled systems contain a reservoir, matrix and porous membrane.

One method for producing sustained delivery of oral medications is to encapsulate active drug with slowly dissolving polymeric materials. The rate of release of active drug is influenced by the thickness and the dissolution rate of the particular polymeric coat of the active drug. By varying the thickness and dissolution rates of coated drug particles in a particular preparation, active drug will be released at different predetermined times. This is generally known as microencapsulation.

Another method for producing sustained or controlled delivery of orally administered drug is the matrix dissolution method. A means for preparing a drug-polymer matrix is “congealing” where the drug is mixed with polymeric substances or waxes. A specific method of congealing is known as “spray-congealing.” Another means for matrix preparation is the aqueous-dispersion method. In this method, the drug-polymer mixture is sprayed or placed in water and then collected.

### EXAMPLE 10

A hybrid of oral administration and osmotic pump delivery is the OROS® system developed by Alza Corporation. In this method, a non-digestible capsule is made of a semi-permeable membrane. Within the confines of this membrane is an osmotic core containing the active drug. As water passes through the semipermeable membrane due to an osmotic gradient, the water tends to push the active drug through an orifice in the capsule. This provides for constant delivery of active drug as the capsule passes through the gastrointestinal system. An example of this system is Acutrim®, which releases phenylpropanolamine in a sustained dose that causes appetite suppression but which produces little, if any, adrenergic-like side effects which typically accompany phenylpropanolamine administration by less controlled bolus administration.

Such non-digestible per os administration of nalmefene may provide a relatively easy solution to sustained action and controlled release of nalmefene consistent with the present invention.

### EXAMPLE 11

Bupropion is co-administered with any of the above examples of nalmefene administration. When administered orally, a sustained release of bupropion is preferred, such as Wellbutrin® SR or Zyban®. Bupropion may be administered in a variety of delivery systems as previously described for nalmefene. A preparation combining the two active drugs nalmefene and bupropion may also be formulated. However, the simple co-administration of an orally administered sustained release tablet of bupropion HCL with nalmefene is suitable for optimizing dopamine release in the central nervous system in the setting of partially blocked mu-opioid receptors.

The invention also provides a method for sterilizing the above-described sustained-release delivery systems. The method comprises exposing a system to sufficient xray radiation to destroy any contaminating microorganisms, without causing any harmful effects to either the active ingredients contained in the sustained-release delivery system or to the composition of the materials comprising the system.

While preferred embodiments have been shown and described, various modifications and substitutions may be made without departing from the spirit and scope of the invention. Accordingly, it is to be understood that the present invention has been described by way of example and not by limitation.

### Material from Ser. No. 10/306,657 (Nov. 27, 2002)—pages 24 thru 49

Hydrogels are polymer networks that swell in water without immediately dissolving. They typically possess good biocompatibility because of low interfacial tension with surrounding biological fluids and the rubbery characteristic that minimizes mechanical and frictional irritation to biological tissue with which they may come in contact. Hydrogels may be made of biodegradable materials and polymers. However, “low-molecular weight water-soluble drugs often permeate hydrogels at too high a rate to be useful” in a non-membrane form [Drug Delivery Systems, p.287, Vasante Ranade and Mannfred Hollinger, ISBN 0-8493-8542-3].

A drug such as nalmefene may be held within a hydrogel composition. Hydrogels may release such a drug by one of several mechanisms. If the hydrogel is a biodegradable polymer, the drug may be released as the polymer is broken down, such as by hydrolysis. Such hydrogels release a drug by erosion of the drug delivery device structure, for example erosion of a polymer network. A second way that the hydrogel may release a drug is that the hydrogel takes in water and swells, as a result of this the network mesh of polymers of which it is made is stretched. Such stretching increases the distance between individual polymers making up the hydrogel network. As this occurs, drug molecules (e.g. nalmefene) may then slip through the individual polymers and diffuse out of the hydrogel network at a rate controlled by the drug's concentration gradient and the distance between individual polymers (which is proportional to swelling of the hydrogel).

Additional factors that effect biodegradation or erosion of the hydrogel in biological systems (in vivo) are the effects of enzymes that are in contact with the hydrogel, and the local inflammatory response that occurs when a foreign body (e.g., a hydrogel) is in contact with biological tissue. For instance, it is well known that many drug delivery devices that are administered to the subcutaneous tissues of an animal or human cause an inflammatory reaction that may, 1) result in phagocytosis or digestion by immunological cells, or 2) result in a deposition of fibrous tissue around the foreign body such that the foreign body will be “walled off” from the immediate subcutaneous tissue outside of the area of the fibrous wall containing the hydrogel. Either of these responses would significantly affect the release rate of drug (e.g. nalmefene) from the hydrogel or foreign body. Thus, incorporation of an anti-inflammatory drug, such as triamcinolone, would directly affect the release rate of the drug from the hydrogel. This affect of anti-inflammatory drugs on drug release rate has not been appreciated by those skilled in the prior art.

Further, because hydrogels typically swell, this may produce hydrostatic pressure against surrounding biological tissue. For example, if a hydrogel is administered to the subcutaneous space in a human, the swelling could cause pressure on the nerves in the subcutaneous tissue resulting in pain. Expected pain would naturally dissuade a human from agreeing to have the hydrogel administered in the subcutaneous space. This also has not been appreciated by those skilled in the art. The inclusion of a local anesthetic drug such as procaine or 2-chloroprocaine would alleviate or prevent the pain as the local anesthetic would be released from the hydrogel and bathe surrounding nerves in the subcutaneous tissue.

It can be important to deliver an active drug such as nalmefene in its hydrochloride salt form because administration of the pure base form of the drug can result in caustic changes in pH in the area immediate to the hydrogel, which can result in irritation and further pain. In addition, the hydrochloride salt form of nalmefene and many other drugs are much more chemically stable than the corresponding pure base forms. Thus, if the goal is to deliver a specific active drug (e.g. nalmefene) into the bloodstream at a given optimally therapeutic rate in order to result in an optimum blood concentration of the drug, the chemical stability of the drug becomes of paramount importance.

An comprehensive review of biodegradable injectable drug delivery systems is given by reference in Journal of Controlled Drug Release 80 (2002), pages 9-20—“Biodegradable injectable in-situ forming drug delivery systems” authored by A. Hatefi and B. Amsden. The present invention differs from those described by Hatefi and Amsden in that the present invention does not form in situ after injection or administration in vivo, but rather is formed ex situ or in vitro. In other words, the present invention is manufactured to completion outside of a living animal or human and maintains its physical characteristics immediately after administration by injection.

The present invention allows for a desirable rate-controlled release of water-soluble molecules from a semi-solid (“semi-sol”) that can be readily injected through a hypodermic needle into subcutaneous tissue. This semi-sol shares some characteristics described above for a hydrogel. It is biodegradable, non-toxic in vivo, and is made of a network of a copolymer. It holds its general shape and form in vivo. However, unlike other drug delivery devices in the prior art that may be injected through a needle and then become a harder discrete form in vivo, the present invention is not based on a solvent diffusing away after subcutaneous administration, and is not based on temperature differences between room temperature and in vivo body temperature, an will maintain its ex vivo or in vitro general form in vivo.

Though the model drug used herein is nalmefene hydrochloride, the present invention in no way is meant to be limited to this particular drug, but rather can have embodiments that encompass many other water-soluble small molecules. When nalmefene is embodied as an active drug of the invention, it may be used preferentially to treat opioid dependence, alcoholism, cocaine dependence, compulsive behaviors such as gambling and sexual addictions, as well as treating HIV-infected individuals to diminish HIV virus activation and replication.

### EXAMPLE 12

A novels moldable drug delivery formulation with putty-like characteristics that releases nalmefene at a desired rate at 37 degrees Celsius and pH of 7.4 is described. The nalmefene-containing formulation may be injected through a typical hypodermic needle to make subcutaneous administration through an 18-gauge needle relatively simple.

Poly(DL-lactide-co-glycolide) copolymers (“PLGA”) with a ratio of lactide to glycolide of 75:25 having an inherent viscosity of 0.69 dL/g in CHCL₃ at 30 degrees Celsius were purchased from Birmingham Polymers, Inc. (Birmingham, Ala.). 2 grams of the PLGA were dissolved in 1 milliliter (“ml”) of the solvent acetone (“solvent 1”). 2 ml of the plasticizing agent triacetin is added to the PLGA-acetone mixture while being stirred. Nalmefene hydrochloride (“HCL”) was purchased from Mallinckrodt, Inc. (St. Louis, Mo.). The nalmefene is dissolved in a solvent (“solvent 2”), examples of which are distilled water and pure ethanol. Low heat, 40-60 degrees Celsius may be used to enhance solubility of nalmefene in the solvent. In one method, the 800 milligrams (“mg”) nalmefene is dissolved in 2 ml (2 grams) of distilled water and 0.575 g PEG 300 at 50 degrees Celsius (“C”). “PEG” is polyethylene glycol, and 300 refers to PEG have specific characteristics. The nalmefene-solvent 2-PEG mixture is then added to the PLGA-solvent1-triacetin mixture with stirring and enough heat to optimally catalyze the mixing or blending of the two mixtures into a new combined mixture. In this instance, temperature in the range of 60-80 degrees C. is used. After stirring to a relatively uniform combined mixture or blend, the combined mixture is placed in a vacuum oven at 60 degrees C. for approximately one hour. The negative pressure within the vacuum oven is adjusted so as to allow solvent 1 and solvent 2 to evaporate without having the combined mixture boil or overflow its container. Alternatively, the combined mixture may be subject to a roto-vaporizor. In this instance, a vacuum oven is utilized as described. The combined mixture is then spread out relatively uniformly on a non-stick surface such as a Teflon® plate and placed once again in the vacuum oven for 30 minutes or more. The soft moldable putty-like material containing nalmefene HCL, triacetin, PEG 300 and PLGA is again manually mixed, stirred or kneaded into a uniform consistency. It is sticky when removed from the oven, but upon cooling the semi-sol delivery blend is easier to manually manipulate and work with. It may be cooled in a water bath. At room temperature is readily moldable and has a putty-like consistency. This moldable semi-sol may then be molded into a tablet configuration, injection molded, or in may be inserted into a syringe and injected through a hypodermic needle. The viscosity of the putty, or its ability to be easily injected through a needle of a specified diameter and length, can be readily manipulated by changing the relative amounts of PLGA, triacetin and PEG. In this instance PEG 300 is used, however PEG 400 or other PEG's may be used.

A moldable semi-sol prepared as just described was put with a phosphate buffer having a pH of 7.4 into a glass vial. The glass vial was then put into a shaker bath at 37 degrees Celsius and release rate of nalmefene were monitored for 27 days. FIG. 3 shows the release rate of nalmefene from three samples. The data show that there is no initial burst of nalmefene and that the rate of release slowly increased over time. These characteristics of nalmefene release are very important when nalmefene is used to treat an opioid dependent human immediately after detoxification or withdrawal. This is because the gradual increase in release of nalmefene allows a relatively smaller amount of nalmefene to be administered soon after detoxification when the human subject is most sensitive to the adverse effects of nalmefene, and allows for a relatively larger, and therefore “more protective,” amount of nalmefene later on as the human subject adapts to not having mu-opioid receptors activated by an opioid agonist. This is expected to produce a much “smoother” time period in the days following detoxification when the nalmefene is administered, such as by subcutaneous injection of the semi-sol sustained deliver system.

Since there was a gradual increase in nalmefene release over the 27 days, the data in FIG. 3 are not absolutely linear. Since the release rate was not absolutely linear, the data was analyzed at 6-day intervals. Since the putty-like semi-sol formed a thin disc in the glass vial (resembling a tidily wink at the bottom of the vial), the release was essentially “one-sided.” Nevertheless, the measured release rate was 0.6 mg/day for the first few days to a maximum of 1.29 mg/day at the near the 27^{th} day (see FIG. 4). The putty swelled with time at a gradual rate. In models described in the prior art, the release rate of a drug or molecule (e.g. nalmefene) in swelling hydrogels is presumed to be diffusion controlled and dependent upon the buffer into the hydrogel. The rate of release would be expected to decrease as the nalmefene became depleted and as the diffusional pathlength increased with swelling. Most unexpectedly, the opposite effect is seen in the present invention, i.e., the rate of release increases with swelling. Thus, the present invention appears to be associated with a new and novel way in which water-soluble molecules are released from a putty-like semi-sol delivery system that swells when in contact with water.

### EXAMPLE 13

The invention generally described above except that the plasticizing agent may be triethyl citrate instead of triacetin.

### EXAMPLE 14

A novels moldable drug delivery formulation with putty-like characteristics that releases buprenorphine at physiological conditions is described. The buprenorphine-containing formulation may be injected through a typical hypodermic needle to make subcutaneous administration through an 18-gauge needle relatively simple.

Poly(DL-lactide-co-glycolide) copolymers (“PLGA”) with a ratio of lactide to glycolide of 75:25 having an inherent viscosity of 0.69 dL/g in CHCL₃ at 30 degrees Celsius were purchased from Birmingham Polymers, Inc. (Birmingham, Ala.). 2 grams of the PLGA were dissolved in 1 milliliter (“ml”) of the solvent acetone (“solvent 1”). 2 ml of the plasticizing agent triethyl citrate is added to the PLGA-acetone mixture while being stirred. Buprenorphine HCl is dissolved in a suitable solvent (“solvent 2”) that evaporates at a temperature and pressure such that the solvent can be readily evaporated without physical breakdown of the buprenorphine core molecule. Low heat, 40-80 degrees Celsius may be used to enhance solubility of buprenorphine in the solvent. In one method, the 800 milligrams (“mg”) buprenorphine is dissolved in 2 ml (2 grams) of distilled water and 0.575 g PEG 400 at 50 degrees Celsius (“C”). The buprenorphine-solvent 2-PEG mixture is then added to the PLGA-solvent1-triethyl citrate mixture with stirring and enough heat to optimally catalyze the mixing or blending of the two mixtures into a new combined mixture. In this instance, temperature in the range of 50-80 degrees C. is used. After stirring to a relatively uniform combined mixture or blend, the combined mixture is placed in a vacuum oven at 60 degrees C. for approximately one hour. The negative pressure within the vacuum oven is adjusted so as to allow solvent 1 and solvent 2 to evaporate-without having the combined mixture boil or overflow its container. Alternatively, the combined mixture may be subject to a roto-vaporizor. In this instance, a vacuum oven is utilized as described. The combined mixture is then spread out relatively uniformly on a non-stick surface such as a Teflon® plate and placed once again in the vacuum oven for 30 minutes or more. The soft moldable putty-like material containing buprenorphine HCL, triethyl citrate, PEG 400 and PLGA is again manually mixed, stirred or kneaded into a uniform consistency. It is sticky when removed from the oven, but upon cooling the semi-sol delivery blend is easier to manually manipulate and work with. It may be uniformly blended at this stage as taffy is manufactured on a “taffy machine.” It may be cooled in a water bath. At room temperature is readily moldable and has a putty-like consistency. This moldable semi-sol may then be molded into a tablet configuration, injection molded, or in may be inserted into a syringe and injected through a hypodermic needle. The viscosity of the putty, or its ability to be easily injected through a needle of a specified diameter and length, can be readily manipulated by changing the relative amounts of PLGA, triacetin and PEG. In this instance PEG 400 is used, however other PEG's may be used.

A 2 milliliter volume of the putty-like semi-sol preparation containing buprenorphine is then put into a 3 cc syringe attached to an 18 gauge needle, the syringe plunger in added air is expelled from the syringe. The syringe containing the putty-like semi-sol preparation containing buprenorphine is then sealed in a package. The sealed package is then subject to enough gamma radiation to effective sterilize the sealed packet with its contents that include the syringe, syringe plunger and the putty-like semi-sol preparation containing buprenorphine. This may now be used for the subcutaneous administration of the sustained drug delivery device by the usual methods.

The characteristics of buprenorphine release from this invention are very important when buprenorphine is used to treat an opioid dependent human that has not been detoxified. This is because the gradual increase in release of buprenorphine allows a relatively smaller amount of buprenorphine to be administered soon after buprenorphine maintenance therapy is initiated when the human subject is most sensitive to the adverse effects of buprenorphine, and allows for a relatively larger, and therefore “more protective,” amount of buprenorphine later on as the human subject adapts to having mu-opioid receptors only partially activated by an partial mu-opioid agonist buprenorphine. This is expected to reduce adverse effects associated with buprenorphine during time period in the days following initiation of buprenorphine maintenance by way of subcutaneous injection of the semi-sol sustained deliver system.

### EXAMPLE 15

The invention generally described in the above examples above except that the active opioid drug is nalbuphine.

### EXAMPLE 16

A novels moldable drug delivery formulation with putty-like characteristics that releases nalmefene at in vitro conditions is described. The nalmefene-containing formulation may be injected through a typical hypodermic needle to make subcutaneous administration through an 18 gauge needle relatively simple.

Poly(DL-lactide-co-glycolide) copolymers (“PLGA”) with a ratio of lactide to glycolide of 75:25 are used. 2 grams of the PLGA were dissolved in 1 milliliter (“ml”) of the solvent acetone (“solvent 1”). 2 ml of the plasticizing agent triacetin is added to the PLGA-acetone mixture while being stirred. Into the PLGA-solvent 1-triacetin mixture, 5 to 10 mg triamcinolone is added. Triamcinolone is commercially available in various pharmaceutical configurations, such as triamcinolone base, triamcinolone acetonide, triamcinolone diacetate, and so forth. A form of triamcinolone that is easily soluble in the PLGA-solvent 1-triacetin mixture used.

Nalmefene hydrochloride (“HCL”) was purchased from Mallinckrodt, Inc. (St. Louis, Mo.). The nalmefene is dissolved in a solvent (“solvent 2”), in this case 2 ml pure ethanol. Low heat, 40-60 degrees Celsius may be used to enhance solubility of nalmefene in the solvent. 800 milligrams (“mg”) nalmefene is dissolved in 2 ml (2 grams) of ethanol and 0.575 g PEG 400 at 50 degrees Celsius (“C”). The nalmefene-ethanol-PEG mixture is then added to the PLGA-solvent1-triacetin-triamcinolone mixture with stirring and enough heat to optimally catalyze the mixing or blending of the two mixtures into a new combined mixture. In this instance, temperature in the range of 60-80 degrees C. is used. After stirring to a relatively uniform combined mixture or blend, the combined mixture is placed in a vacuum oven at 60 degrees C. for approximately one hour. The negative pressure within the vacuum oven is adjusted so as to allow solvent 1 and solvent 2 to evaporate without having the combined mixture boil or overflow its container. Alternatively, the combined mixture may be subject to a roto-vaporizor. In this instance, a vacuum oven is utilized as described. The combined mixture is then spread out relatively uniformly on a non-stick surface such as a Teflon® plate and placed once again in the vacuum oven for 30 minutes or more. The soft moldable putty-like material containing nalmefene HCL, triamcinolone, triacetin, PEG 400 and PLGA is again manually mixed, stirred or kneaded into a uniform consistency. It is sticky when removed from the oven, but upon cooling the semi-sol delivery blend is easier to manually manipulate and work with. It may be cooled in a water bath. At room temperature is readily moldable and has a putty-like consistency. This moldable semi-sol may then be molded into a tablet configuration, injection molded, or in may be inserted into a syringe and injected through a hypodermic needle. The viscosity of the putty, or its ability to be easily injected through a needle of a specified diameter and length, can be readily manipulated by changing the relative amounts of PLGA, triacetin and PEG. In this instance PEG 400 is used, other PEG's may be used. Further, other PLGA's may be used such as PLGA 65:35, PLGA 60:40, PLGA 85:15, etc.

The added advantage of this embodiment is that the semi-sol putty will be more resistant to phagocytosis induced by a foreign body reaction, thus leaving the putty intact in vivo long enough for the nalmefene to diffuse out of the putty. After the greater portion of the nalmefene leaves the putty, the putty is then left for bio-erosion or biodegradation. If however, the putty is degraded so fast such that the greater portion of nalmefene is still within it upon erosion, then a significant drug burst (of nalmefene) can be expected. Thus, the triamcinolone is an important component of the invention for in vivo use, which contrasts to the in vitro data depicted in FIGS. 3 and 4. This effect of triamcinolone in preventing drug burst and allowing for a continued release rate of nalmefene that is not drastically accelerating, has not been appreciated in the prior art. Triamcinolone is the preferred steroidal anti-inflammatory drug because it tends to have a more local effect on the immediately surrounding tissue and less of a systemic effect throughout the body, as compared to some of the other steroidal anti-inflammatory drugs.

### EXAMPLE 17

The invention generally described in the above examples above except that an active water-soluble drug delivered in sustained release fashion with the characteristics described is a salt form of naltrexone such as naltrexone hydrochloride.

### EXAMPLE 18

The invention generally described in the above examples above except that an active water-soluble drug delivered in sustained release fashion with the characteristics described is a salt form of 6-methyl-naltrexone such as 6-methyl-naltrexone hydrochloride.

### EXAMPLE 19

The invention generally described in the above examples above except that an active water-soluble drug delivered in sustained release fashion with the characteristics described is a water-soluble salt form of 6-beta-naltrexol.

### EXAMPLE 20

The invention generally described in the above examples above except that an active water-soluble drug delivered in sustained release fashion with the characteristics described is a kappa-opioid receptor selective antagonist such as nor-binaltorphimine (“nor-BNI”).

### EXAMPLE 21

The invention generally described in the above examples above except that an active water-soluble drug delivered in sustained release fashion with the characteristics described is a salt form of a kappa-opioid receptor selective agonist such as U50,488 or enadoline.

### EXAMPLE 22

The invention generally described in the above examples above except that an active water-soluble drug delivered in sustained release fashion with the characteristics described is a salt form of a delta-opioid agonist such as benzamide, SNC-80 or deltorphin.

### EXAMPLE 23

Poly(DL-lactide-co-glycolide) copolymers (“PLGA”) with a ratio of lactide to glycolide of 50:50 having an inherent viscosity of 0.59 dL/g in HFIP at 30 degrees Celsius were purchased from Birmingham Polymers, Inc. (Birmingham, Ala.). 2 grams of the PLGA were dissolved in 2 milliliter (“ml”) of the solvent acetone (“solvent 1”). 1 ml of the plasticizing agent triacetin is added to the PLGA-acetone mixture while being stirred. Nalmefene hydrochloride (“HCL”) was purchased from Mallinckrodt, Inc. (St. Louis, Mo.). The nalmefene is dissolved in a solvent (“solvent 2”), examples of which are distilled water and pure ethanol. Low heat, 40 degrees Celsius may be used to enhance solubility of nalmefene in the solvent. 500 milligrams (“mg”) nalmefene is dissolved in 1 ml (1 grams) of distilled water and 0.420 g PEG 300 at 40 degrees Celsius (“C”). The nalmefene-solvent 2-PEG mixture is then added to the PLGA-solvent1-triacetin mixture with stirring and enough heat to optimally catalyze the mixing or blending of the two mixtures into a new combined mixture. In this instance, temperature in the range of 45-65 degrees C. is used. After stirring to a relatively uniform combined mixture or blend, the combined mixture is placed in a vacuum oven at 60 degrees C. for approximately one hour. The negative pressure within the vacuum oven is adjusted so as to allow solvent 1 and solvent 2 to evaporate without having the combined mixture boil or overflow its container. Alternatively, the combined mixture may be subject to a roto-vaporizor. In this instance, a vacuum oven is utilized as described. The combined mixture is then spread out relatively uniformly on a non-stick surface such as a Teflon® plate and placed once again in the vacuum oven for 30 minutes or more. The soft moldable putty-like material containing nalmefene HCL, triacetin, PEG 300 and PLGA is again manually mixed, stirred or kneaded into a uniform consistency. At room temperature it is readily moldable and has a putty-like consistency without the need for additional cooling. This moldable semi-sol is molded into a tablet configuration weighing out 0.700 g of the final putty and pressing it into a circular mold with the dimension of 15 mm diameter by 3 mm depth. Alternatively, this putting is easily injection molded to the shape and size desired. The nalmefene drug loading for each tablet is 12%.

A moldable semi-sol tablet prepared as just described was put with 10 ml phosphate buffer having a pH of 7.4 into a 20 ml scintillation via into which glass wool was previously laid. After three days, the glass wool was replaced with a wire mesh, and on day 6 with Teflon®. The buffer was replaced with 10 ml of fresh solution after each measurement of the concentration of nalmefene. The measured nalmefene concentration was measured against a standard curve using know concentrations of nalmefene. The cumulative weight of nalmefene was plotted versus time. The nalmefene release rate was calculated by determining the slope of the curve at a given time interval. FIG. 5 depicts a graph of the release rate of nalmefene from three samples of the PLGA 50:50 blended putty containing 86 mg of nalmefene over time. FIG. 6 is data on nalmefene release rates at time intervals from 0-5 days, 5-10 days, 10-16 days, 16-20 days and 20-23 days. The data show that there is no initial burst of nalmefene and that the rate of release slowly increased over time. These characteristics of nalmefene release are very important when nalmefene is used to treat an opioid dependent human immediately after detoxification or withdrawal. This is because the gradual increase in release of nalmefene allows a relatively smaller amount of nalmefene to be administered soon after detoxification when the human subject is most sensitive to the adverse effects of nalmefene, and allows for a relatively larger, and therefore “more protective,” amount of nalmefene later on as the human subject adapts to not having mu-opioid receptors activated by an opioid agonist This is expected to produce a much “smoother” time period in the days following detoxification when the nalmefene is administered, such as by subcutaneous injection of the semi-sol sustained deliver system.

The putty swelled with time at a gradual rate. The PLGA 50:50 putty blend swelled to only, taking in a maximum amount of water from the buffer solution by day 10, and the amount of swelling was only 60% of the original volume. This represents a maximum increase in size of 1.6 times, a volume that would be well tolerated by gradual swelling when administered into subcutaneous tissue. The tablet began to disintegrate after several weeks. This is desirable because the biodegradable implant is intended for complete absorption in vivo, thus removal of a drug delivery device shall not be required after the nalmefene is released.

Using various blends, it was determined by experimentation that degradation occurred with PLGA 50:50 putty blends after several weeks, with PLGA 65:35 blends after a slightly longer period, and degradation was not seen with PLGA 75:25 blends up through over four weeks. Bioerosion therefore is predicted to be proportional to the amount of glycolic acid in the PLGA co-polymer. The steroidal anti-inflammatory drug triamcinolone would tend to decrease faster degradation due to bioerosion by inhibiting the inflammatory response, therefore ensuring relatively prolonged release of the active water-soluble drug molecule (e.g. nalmefene). Further, the present invention teaches that release rate can be maintained at higher levels by minimizing thickness of the mold or putty-like sample (“putty”), or increasing its surface area relative to its volume. For example, the tablets just described were 3 mm thick. Cylinders formed by extrusion of the putty through a hollow needle would yield such a result. Even the more viscous putty with one-half the ratio of triacetin is easily formed into various physical shapes by the process of extrusion. Triacetin is a preferred plasticizer because it has been used parenterally in animals without adverse effects, and has been approved for human use as a pharmaceutical plasticizer for non-parenteral uses. For example, Tarr, Sambandam and Yalkowsky writing in “Pharm Res April 1987;4(2):162-5” in an article titled “A new parenteral emulsion for the administration of Taxol” teach that emulsions for drug delivery made of 50% triacetin can be safely injected intravenously into mice. Bailey, Heath and Miles in “Am J Clin Nutr February 1989;49(2):385-8” teach that the short chain triglyceride triacetin may be used as a parenterally administered nutrient with no demonstrable adverse effects on calcium, magnesium or phosphorus metabolism in dogs (see abstract “Calcium, magnesium, and phosphorus metabolism in dogs given intravenous triacetin”). Bailey, Haymond and Miles in “JPEN J Parenter Enteral Nutr January-February 1991;15(1):32-6 write in “Triacetin: a potential parenteral nutrient” that triacetin is safely infused intravenously in dogs. Karlstad, Killeffer, Bailey and DeMichele in “Am J Clin Nutr May 1992;55(5):1005-11” teach that triacetin as a component of total parenteral nutrition, also known generally as “TPN,” is safe and effective in rats. Likewise, Bleiberg, Beers, Persson and Miles writing in “Am J Clin Nutr December 1993;58(6):908-11” teach that triacetin may be used as a form of intravenous nutrition in dogs. Subsequent studies have shown similar utility of triacetin. To this author's knowledge, the present invention is the first in which triacetin is used as a plasticizer for in vitro or ex vivo formation of a pharmaceutical delivery system intended for subcutaneous administration. Thus, this too represents a novel, unique and unobvious component of the present invention.

The above examples are for illustrative purposes only, and are not intended to limit the scope of the invention in any way. In light of the present invention that was brought about by experimentation, and to some degree by fortuitous circumstance, other embodiments of the invention may be apparent to those skilled in the art. Such embodiments may come after additional experimentation, but nonetheless experimentation that is usual and typical of the kind carried out in formulating and testing pharmaceutical preparations. Such examples have been mentioned herein, such as altering the PLGA used, altering the ratio of a particular PLGA to a particular plasticizer, etc. Also the concentration and ratio of active drugs, such as nalmefene, buprenorphine, nalbuphine and other water soluble drugs of similar molecular weight, ionic charge and so forth, can be manipulated to yield a drug release that would be optimal for that particular active drug based on characteristics of the particular drug such as it's molecular structure, volume of distribution, lipid solubility, elimination half-life, biological half-life, pK, pH of the surrounding medium, physical stability of the drug in a medium, etc. The release rate of active water-soluble drug has also been found able to be altered by limiting or eliminating PEG from the final product, as PEG because of its physical characteristics promotes swelling. The present invention also teaches that blending PLGA 75:25 copolymer with PLGA 50:50 copolymer may limit swelling compared to 75:25 PLGA alone, while limiting degradation compared to PLGA 50:50 alone. What is novel and new is the mechanism of release of drug, along with its resultant characteristics relating to rate of drug release, rate of change of drug release, lack of drug burst, and the way these characteristics may relate to the disease entities for which the active drugs are intended to treat. It is of further great utility that administration by simple subcutaneous needle injection is easy, practical and does not involve more invasive surgical procedures such as formed pellet implantation. Further embodiments of the invention will become apparent in light of the teachings that follow.

Gekker, Lokensgard and Peterson in Drug and Alcohol Dependence 64 (2001), pages 257-263 describe in “Naltrexone potentiates ant-HIV1 activity of antiretroviral drugs in CD4+ lymphocyte cultures” how naltrexone acts to increase the anti-HIV effects of drugs used to treat H[V infection and AIDS. Li, Wang, Tian et al. in Journal of Infectious Disease, January 1;185(1):118-22 teach in “Methadone enhances human immunodeficiency virus infection of human immune cells” that the opioid agonist methadone increase HIV virus activation and replication. These authors reiterate their findings in the Journal of Infectious Disease Jan. 1, 2002;185(1):118-22 in another article with the same title. Mahayni and Minor wrote a letter in the American Journal of Hospital Pharmacy Nov;48(11):2480-1 stating that “research data suggests that the narcotic antagonists naltrexone and naloxone may possess anti-HIV activity,” but they make no mention of nalmefene. Dr. Bernard Bihari wrote a letter in AIDS Patient Care February 1995;9(1):3 observing that trials of low-dose naltrexone with AIDS patients showed that the naltrexone was associated with significant and advantageous differences in the incidence of opportunistic infections, and that AIDS patients administered naltrexone maintained “good” CD4 lymphocytes. He intimates that this may be due to the immunological role of endorphins as “key hormones” in regulating the immune system. Schluger, Ho, Borg et al. in Alcohol Clinical Experimental Research October 1998;22(7):1430-6 in their article “Nalmefene causes greater hypothalamic-pituitary-adrenal activation than naloxone in normal volunteers: implications for treatment of alcoholism” demonstrate that “kappa- and delta-opioids may play important roles in the regulation of the hypothalamic-pituitary-adrenal axis.” (They do not discuss any possible role related to HIV). Suzuki, Chuang, Chuang et al. in Advances in Experimental Medicine and Biology 2001;493:81-7 in a chapter titled “Morphine upregulates kappa-opioid receptors of human lymphocytes” teach that “chronic morphine use also induces immunomodulatory and immunosuppressive effects, as especially evident in HIV-infected patients,” and that this phenomenon involves kappa-opioid receptors. This, taken together with the advantageous opioid receptor subtype binding profile of nalmefene as described herein and which was noted prior to October 1998 in U.S. patent application Ser. No. 08/643,775 filed May 6, 1996 supports the present invention that teaches nalmefene as a preferential treatment and prophylactic medication for HIV viral infection and AIDS. The present invention also teaches the manufacture and use of a pharmaceutical preparation for preventing HIV infection as a prophylactic measure and for treating HIV infection in HIV-infected individuals and those with AIDS.

The present invention also teaches the superiority of nalmefene for treatment of alcoholism compared to other opioid antagonists due to a decreased tendency for cardiac dysrhythmias in alcoholic patients at increased risk for such dysrhythmias that was not previously appreciated by those skilled in the art. Nalmefene has been used to treat alcoholism, most notable by Dr. Barbara Mason in Florida. However, it has really been used in the prior art as an analogous compound to naltrexone, which the invention at hand clearly demonstrates it is not. In addition to the distinguishing characteristics of nalmefene demonstrated previously herein, as it relates to a drug addiction when the drug abused is ethanol (“alcohol”) the following advantages of nalmefene are encompassed in the present invention. Smetnev, Gorgaaslidze, Zinkin et al. in “Terk Arkh 1988;60(1):49-51” [orginial article in Russian], point out that more than 29% of alcoholic patients have cardiac abnormalities manifesting as “arrhythmical paroxysms.” Faintuch in “Rev Hosp Clin Fac Med Sao Paulo January-February 1995;50(1):76-9” [original article in Portuguese] states that “both acute and chronic alcohol consumption precipitate arrhythmias.” Fabrizio and Regan in “Cardiovasc Drugs Ther February 1994;8(1):89-94 report in their article “Alcoholic cardiomyopathy” that “atrial arrhythmias have been shown to occur during the early ethanol withdrawal phase in patients without other clinical evidence of heart disease.” It is common knowledge in medical practice that “holiday heart syndrome” consists of cardiac dysrhythmia due to the high ingestion of ethanol around the time of holiday celebrations, which quite unfortunately is sometimes fatal. Actions of alcohol inducing dysrhythmias on a cellular level have been described in animal models. For instance, Nakamura, Houchi, Ohe an Namba in Alcohol Clinical Experimental Research April 1999;23(4 Suppl):81S-84S teach in their article “Increase in beating rate of cultured chick cardiac myocytes by ethanol and inhibition of the increase by antiarrhythmic drugs” that “drinking alcohol sometimes causes cardiac arrhythmia.” Going back to 1976, Ettinger et al. teach in the American Heart Journal January 1976;91(1):66-78 in their article “Cardiac conduction abnormalities produced by chronic alcoholism” that “cardiac conduction abnormalities and rhythm disturbances are common clinical findings” in alcoholic patients with manifestations of long-term alcohol consumption. Those skilled in the art of treating alcoholic human patients clinically have failed to appreciate what is demonstrated by Caldwell, Nagarajan, Chryssanthis and Tuttle in Pharmacology 1990;41(3):161-6 in their articled titled “Actions of the opioid antagonist, nalmefene, and congeners on reperfusion cardiac arrhythmias and regional left coronary blood flow.” Caldwell et al. reach that nalmefene “reduced the incidence of reperfusion arrhythmias significantly when compared to the saline control,” and that “neither N-methyl-nalmefene . . . nor (+)nalmefene . . . provided any protection against reperfusion arrhythmias.” They concluded that nalmefene prevents the occurrence of such arrythmias. These studies, taken within the context of treating human alcoholics with opioid antagonists, support the present invention that nalmefene, as distinguished from other opioid antagonists used in treatment of drug addiction (e.g. naltrexone), is a preferred drug in the treatment of alcoholic patients. This, not being obvious to others skilled in the art, has not been attributed to nalmefene in the context of clinical trials using nalmefene to treat alcoholism. The present invention teaches that nalmefene, being non-analogous to naltrexone, is a preferred drug for the treatment of alcoholism. Schluger et al (ibid) do not make the case for nalmefene as being a preferred drug for treatment in alcoholism. In fact, they state merely “the effects of nalmefene and also naltrexone on modulating the tonic inhibition exerted by endogenous opioids acting at kappa- and delta-, as well as mu-, opioid receptors on the hypothalamic-pituitary-adrenal (“HPA”) axis may be related to their [emphasis added] established efficacy as treatment agents for alcoholism (see page 1434, Ibid). Further, with regard to nalmefene being a non-analogous preferred agent to naltrexone for alcoholism, Schluger et al. only conclude “nalmefene, as well as other kappa and perhaps delta-opioid antagonists and agonists, may therefore be useful tools to further elucidate some of the basic physiology and pathophysiology of the HPA axis, the endogenous opioid system, the biology of addictions, and the intersections between them” (see page 1435,]bid). Such vague and convoluted language does not teach nalmefene as a preferred opioid antagonist to treat alcoholism, therefore the present invention is not obvious to one of ordinary skill in the art because of Schluger et al.

The present invention also teaches that 6-beta-naltrexol is a preferred opioid antagonist for the treatment of addictions, most notably opioid addiction, as is nalbuphine. Wang, Raehal, Blisky and Sadee in their article “Inverse agonists and neutral antagonists at mu opioid receptor (MOR): possible role of basal receptor signaling in narcotic dependence” in the Journal of Neurochemistry June 2001;77(6):1590-600, teach that the neutral opioid antagonist 6-beta-naltrexol possesses certain important advantages over other opioid antagonists in that 6-beta-naltrexol does not inhibit intrinsic mu-opioid receptor “agonist-like activity” to the degree that the opioid antagonists naltrexone, naloxone and nalmefene do. This is important in a previously opioid-dependent patient recently detoxified and withdrawn from opioids to which a sustained release opioid antagonist is administered. Clinical evidence indicates that naltrexone pellets implanted into humans as part of a detoxification procedure is associated with substantial withdrawal related signs and symptoms in the post-detoxification period. Accordingly, 6-beta-naltrexol would not cause the same degree of opioid withdrawal related symptoms in the post detoxification period if it were administered in lieu of naltrexone.

The present author has previously quite unexpectedly discovered that nalbuphine, also known by the trade name Nubain® (Endo Pharmaceuticals, Chadds Ford, Pa.), reverses detrimental effects of fentanyl such as respiratory depression without totally inhibiting mu-opioid agonist-like activity. The present author has also quite surprising discovered that nalbuphine, although precipitating opioid withdrawal in actively opioid-dependent patients due to antagonist effects, also relieves opioid withdrawal symptoms in patients in the immediate post-detoxification period. Thus, by incorporating nalbuphine hydrochloride in a putty-like semi-sol described herein for administration and sustained delivery of the water-soluble nalbuphine molecule, a method of blocking opioid receptors from “pure” opioid agonist analgesics, such as morphine, heroin or fentanyl is manifested, while simultaneously allowing for some mu-opioid agonist-like activity. Thus, the present invention recognizes nalbuphine as a partial mu-opioid agonist, which may be interpreted that it is also a partial mu-opioid antagonist, and at the very least allows for intrinsic mu-opioid receptor activity.

The present invention also provides a structural composition comprising a therapeutic dose of opioid agonist analgesic in combination with an amount of 6-beta-naltrexol or nalbuphine effective to allow for the positive effects of the opioid agonist analgesic, while at the same time exerting relatively less antagonistic effects at mu-opioid receptors compared to other opioid antagonists, when the opioid agonist analgesic is administered in recommended therapeutic doses, such that the agonist actions of the opioid agonist analgesic will far outweigh any antagonism by 6-beta naltrexol or nalbuphine at said mu-opioid receptors. If excessive amounts of the structural composition comprising 6-beta naltrexol or nalbuphine and opioid agonist analgesic are administered, enough 6-beta naltrexol or nalbuphine shall be administered as to begin to antagonize or block mu-opioid receptors from the exogenously administered opioid agonist analgesic, while simultaneously allowing for some intrinsic mu-opioid agonist-like activity. Thus, excessive opioid agonist analgesic is blocked, preventing overdose or excessive euphoric effects, while the likelihood of triggering a withdrawal response is greatly diminished.

### EXAMPLE 24

A recommended therapeutic dose of morphine, e.g. 0.15 mg/kg morphine, preferably in the form of morphine sulfate, is co-administered parenterally with 0.00025 to 0.0015 milligrams per kilogram (mg/kg) 6-beta naltrexol, preferably in the form of 6-beta naltrexol hydrochloride, more preferably 0.0007 mg/kg 6-beta naltrexol. For a young adult 70 kg human, for example, 10.5 mg morphine sulfate is administered parenterally, along with 0.049 mg, or 49 micrograms (ug), 6-beta naltrexol hydrochloride parenterally. This small amount of 6-beta naltrexol, consistent with the present invention, produces minimal appreciable effect at mu-opioid receptors in relation to the 10.5 mg dose of morphine.

In a preferred embodiment of the present invention, morphine sulfate and 6-beta naltrexol hydrochloride are co-existent in a common medium compatible for parenteral administration in the ratio, of 0.15 mg active morphine to 0.0007 mg active 6-beta naltrexol. Ideally, if administered subcutaneously, the total amounts of the two co-administered active drugs would be contained within an injectable volume of approximately 1 to 2 milliliters (cc) or less for a 70 kg adult human.

### EXAMPLE 25

Among the most commonly written prescriptions in the United States, are the prescriptions for combination oral analgesics consisting of an opioid agonist analgesic and a non-opioid analgesic such as acetaminophen, aspirin, ibuprofen or other non-steroidal anti-inflammatory drug (“NSAID”). By way of example only, the combination of oxycodone and acetaminophen, commonly known by the brand name Percocet®, is very often prescribed for a wide variety of pain syndromes, including pain secondary to surgery or trauma, and malignancies. Similarly, the drug formulation commonly known by the brand name Percodan® is composed of oxycodone and aspirin, and the opioid agonist analgesic hydrocodone in its bitartrate form is combined with the non-opioid analgesic acetaminophen.

Orally administered combination drugs consisting of an opioid agonist analgesic and another drug(s) or medication(s) are among the most widely abused opioid agonists abused. If these combination drugs contain acetaminophen, as in the case with Percocet®, a large amount of Percocet® tablets may be orally ingested, so much so as to cause a toxic load of acetaminophen to be delivered. Acetaminophen is widely known to be toxic to the liver of humans when administered in excessive dosages, or when abused by self-administration either intentionally or unintentionally. Often, because of the tolerance built up to the opioid agonist analgesic component of the Percocet®, the patient will progressively ingest more and more Percocet® tablets over time in an attempt to satisfy the effect of the opioid agonist analgesic at mu opioid receptors. Because of the stealth adverse effects of acetaminophen toxicity, often a human may not be aware of harm caused to him or her by the large ingestion of acetaminophen in the combination drug formulation, until a medical exam reveals abnormal liver function, or until liver failure suddenly becomes apparent. Compounding this problem is the fact that orally administered drugs are delivered relatively directly to the liver, by a mechanism commonly called “first pass metabolism.” Thus, there is presently a great need to formulate an orally administrable combination drug therapy that would allow for the additive or synergistic effects of combining analgesics, such as opioids and acetaminophen, but that could prevent or strongly limit the likelihood of developing liver failure secondary to liver toxicity.

In the case of Percodan®, a large amount of Percodan® tablets may be orally ingested, so much so as to cause a toxic load of aspirin to be delivered. Aspirin and other NSAIDs are widely known to cause gastrointestinal bleeding of humans when administered in excessive dosages. Often, because of the tolerance built up to the opioid agonist analgesic component of the Percodan®, the patient will progressively ingest more and more Percodan® tablets over time in an attempt to satisfy the effect of the opioid agonist analgesic at mu opioid receptors. Because of the unobvious early signs of adverse effects of aspirin or NSAID toxicity, often a human may not be aware of harm caused to him or her by the large ingestion of aspirin or NSAID in the combination drug formulation, until a medical exam reveals abnormal gastric function, or until an acute gastric hemorrhage suddenly becomes apparent. Compounding this problem is that when aspirin or other NSAIDs are administered orally, or per os, they are delivered directly to the stomach through the esophagus where their breakdown begins to occur in direct contact with the gastric mucosa lining the stomach. This is the very site of the caused gastric bleeding. Thus, there is presently a great need to formulate a orally administrable combination drug therapy that would allow for the additive or synergistic effects of combining analgesics, such as opioids and NSAIDs (of which aspirin is an example), but that could prevent or strongly limit the likelihood of developing gastrointestinal bleeding.

Hydrocodone (as hydrocodone bitartrate, for example) and other opioid agonist analgesics are commonly mixed with other non-opioid analgesic drugs in formulating combination medications.

By way of example only, if a combination medication tablet that is formulated with 10 milligrams (mg) of hydrocodone and 0.2 mg nalbuphine (=200 micrograms) is ingested orally, only about 30 microgram (mcg) of nalbuphine will be delivered unmetabolized to the bloodstream. A formulation proportionate to 10 mg hydrocodone and 100 mcg nalbuphine (0.1 mg), perhaps mixed with acetaminophen 500 mg, would be expected to produce analgesia not significantly or very appreciably different from a formulation of 10 mg hydrocodone and 500 mg acetaminophen without nalbuphine. However, if a human were to ingest two such formulated tablets every four hours, as commonly occurs when human patients self-administer these medications in larger than doses prescribed or intended by a physician, then over a 12-plus hour period a human would ingest 8 tablets comprising 80 mg hydrocodone and 0.8 mg nalbuphine. Because the plasma half-life of both hydrocodone and acetaminophen is approximately 3 hours, and the plasma elimination half-life of nalbuphine is approximately 3.5 hours, nalbuphine will tend to accumulate over time relative to hydrocodone and acetaminophen such that as more time progressively transpires the nalbuphine serum concentration relative to hydrocodone serum concentration will increase as the tablets are ingested over that time. Further, by administering the medication every 4 hours, steady state concentration of nalbuphine will occur in approximately 24 hours. Eventually, this will cause an appreciably different effect of the opioid agonist analgesic. This effect could include prevention of mortal respiratory depression, or lack of satisfaction due to opioid ingestion. The exact nature of this interaction is easily altered by one skilled in the art by changing the relative amounts of nalbuphine to opioid agonist analgesic in the tablet, as well as by altering the pharmacokinetic profile of either drug by including a sustained release preparation of either the nalbuphine or the opioid agonist analgesic. This would be calculated during the normal course of experimentation routine for deriving such data. Such experimentation is routinely employed in formulating pharmaceutical preparations to required standards. The dosages are thus described generally in terms of pharmacological effect.

Though hydrocodone and oxycodone are mentioned by way of example here, the scope of the invention encompasses any orally administered opioid agonist analgesic. Such applicable opioid agonist analgesics include the following opioids and their derived salts and bases: morphine, propoxyphene, fentanyl, methadone, levomethadyl (LAAM) and codeine.

### New Material—pages 50 thru 102

Definitions:

“Opioid antagonist” not qualified by the words “neutral” or “preferred, for the treatment of addictions”=“inverse opioid agonist”=“inverse agonist” is a compound, molecule or peptide that binds to an opioid receptor causing a tendency toward activation or excitation of the opioid receptor, meaning an increase in the signaling from the receptor to endogenous cellular systems upon which the signals from the opioid receptor act, e.g. resulting in an increase in intracellular cAMP, and shall include the base, pharmaceutically acceptable salts thereof, stereoisomers thereof, ethers and esters thereof, and mixtures thereof.

“Neutral opioid antagonist”=“neutral receptor binding agent”=“neutral antagonist” is a compound, molecule or peptide that binds to an opioid receptor without causing a tendency toward activation or inactivation of the opioid receptor different from the state of activity that the opioid receptor is in at the time of binding to it by the neutral receptor binding agent, meaning that signaling from the opioid receptor to endogenous cellular systems upon which the signals from the opioid receptor act are not effectively increased or decreased, e.g., intracellular cAMP is relatively unchanged, and shall include the base, pharmaceutically acceptable salts thereof, stereoisomers thereof, ethers and esters thereof, and mixtures thereof.

“Preferred opioid antagonist for the treatment of addictions” is an opioid binding agent that is not an inverse opioid agonist at mu₁ opioid receptors in individuals that are dependent or addicted to any opioid agonist analgesic.

“Opioid agonist”=“opioid agonist analgesic” is a compound, molecule or peptide that binds to an opioid receptor causing a tendency toward inactivation of the opioid receptor, meaning an decrease in the signaling from the receptor to endogenous cellular systems upon which the signals from the opioid receptor act, e.g., resulting in a decrease in intracellular cAMP. The term “opioid agonist” shall include the base of the opioid, pharmaceutically acceptable salts thereof, stereoisomers thereof, ethers and esters thereof, and mixtures thereof.

“Efficacy” relates to the degree of signaling from the receptor to endogenous cellular systems upon which the signals from the opioid receptor act. For example, high efficacy would mean, e.g., that upon binding to the receptor by an agonist compound, molecule or peptide, a high degree of inhibition of adenyl cyclase occurs in association with relatively lower intracellular concentration(s) of cAMP. Likewise, low efficacy would mean, e g., that upon binding to the receptor by the agonist compound, molecule or peptide, adenyl cyclase inhibition is of a relatively low degree in association with relatively less of a decrease in intracellular concentration(s) of cAMP. There may be a spectrum of efficacies among different compounds that bind to opioid receptors.

“Potency” is the affinity to which the compound, molecule or peptide binds to the receptor. Sometimes, the reciprocal of affinity is expressed as a dissociation constant (K_{D}). A given compound, molecule or peptide may have a high potency and a high efficacy, or a high potency and a low efficacy, or a low potency and a high efficacy or a low potency and a low efficacy, or various graduations of efficacy and potency.

“Partial mu-opioid agonist” is a compound, molecule or peptide that has an efficacy lower than a “full” mu-opioid agonist. The terms “opioid agonist” and “partial” opioid agonist are often quoted in the literature as terms relative to one another. For example, morphine is often cited simply as a mu-opioid agonist, and compared to it, buprenorphine is a partial mu-opioid agonist because of its lower efficacy. Generally, buprenorphine is thought to have a higher potency and a lower efficacy compared to morphine.

“Intrinsic mu-receptor activity” is the amount of signaling from the receptor to endogenous cellular systems upon which the signals from the opioid receptor act, that result when the mu-receptor is not acted upon by an opioid agonist, partial mu-opioid agonist or an opioid antagonist. It is theoretically thought of as the amount of signaling of an unbound or “empty” receptor that is not conformationally altered from its natural resting state under a given set of environmental or historical circumstances.

“Any licit use” of an opioid preparation means to use an opioid for a prescribed or otherwise legitimate and legal medical reason, such reasons including but not limited to the medical treatment of cough, loose stool or diarrhea (including as associated with irritable bowel syndrome), shivering, dependency or addiction, heart disease and pain (including as associated with fibromyalgia, cancer, arthritis, traumatic injury, neuralgia, somatic pain, visceral pain, neuropathic pain, etc.).

The term “parenterally” as used herein includes subcutaneous injections, intravenous, intramuscular, intrastemal injection or infusion techniques.

The term “effective analgesia” is defined for purposes of the present invention as a satisfactory reduction in or elimination of pain, along with a tolerable level of side effects, as determined by the human patient.

The term “sustained release” is defined for purposes of the present invention as the release of the drug (opioid analgesic) from the oral formulation at such a rate that blood (e.g., plasma) concentrations (levels) are maintained within the therapeutic range (above the minimum effective analgesic concentration or “MEAC”) but below toxic levels over a period of time indicative of a twice-a-day or a once-a-day formulation.

The term “steady state” refers to a time when the rate of elimination of a drug is the same as the rate of absorption of that drug into the body.

To put the above-described definitions in a context that better explains them, consider the research article “Activity of opioid ligands in cells expressing cloned mu opioid receptors” by Gharagozlou, et al. in BMC Pharmacology 2003, 3:1. In this work, efficacies and potencies of various mu-opioid acting compounds were determined relative to the naturally occurring beta-endorphin and the common opioid agonist analgesic morphine, by determining how much cAMP was released (expressed in terms of IC50), and by assigning an affinity constant (Kᵢ) a given compound, respectively. For further clarification, 6-beta-naltrexol is a neutral receptor binding agent and nalbuphine is a partial mu-opioid agonist under applicable environmental and historical conditions.

For completeness sake, it should be understood that activation of mu and delta opioid receptors open potassium channels, which decreases calcium conductance. The activation of kappa opioid receptors also reduces calcium channels, but by the mechanism of directly closing the calcium channels. Calcium conductance can be measured by recording an action potential resulting from movement of calcium ions. Excitation or inhibition of the calcium current is sometimes represented by a change in action potential duration.

### OBJECTIVES OF THE INVENTION

It is an object of the invention to provide an oral or parenteral dosage form of an opioid analgesic which is subject to less abuse potential via the oral route than prior commercially available dosage forms that is more humanely administered with less likelihood of a serious or life-threatening adverse event compared to technology in the prior art.

It is a further object of the present invention to provide an oral or parenteral dosage form of an opioid analgesic and method which provides therapeutic analgesia and which also provides a limit on the agonist effects of the opioid analgesic such that a human subject would not be motivated to take much more, e.g. about 2-3 times more than the prescribed dose, of opioid analgesic for any licit use.

It is a further object of the present invention to provide an oral or parenteral dosage form of an opioid analgesic that in non-dependent or opioid naive individuals will not result in any appreciable withdrawal effect and will still allow effective analgesia in opioid naïve individuals even when administered at much more than the prescribed dose, while tending to negate excessive opioid agonist effects when administered at much more than the prescribed dose in opioid-dependent or opioid addicted individuals.

It is a further object of the present invention to provide an oral or parenteral dosage form of an opioid analgesic and a method for providing therapeutic analgesia in a manner which is not as positively reinforcing in opioid naive individuals or humans that are not opioid-dependent taking much more than the prescribed dose, e.g., at least about 2-3 times the prescribed dose of the opioid, as compared to the same amount of opioid without the neutral receptor binding agent or partial mu-opioid agonist.

It is a further object of the invention to provide a method of treating pain in human patients with an oral or parenteral dosage form of an opioid analgesic while reducing the abuse potential of dosage form.

It is a further object of the invention to provide a method of manufacturing an oral dosage form of an opioid analgesic such that it has less oral abuse potential.

### SUMMARY AND FURTHER BACKGROUND

The present invention provides for a pharmaceutical composition comprising an opioid agonist, a neutral receptor binding agent or a partial mu-opioid agonist, in a pharmaceutically acceptable carrier thereof. This is of great societal importance because the invention tends to limit opioid effects to those intended or prescribed for any licit use while further improving upon previous technology by i) providing for a means of differentiating effects of an analgesic composition in opioid dependent versus non-dependent individuals, and ii) decreasing the likelihood of precipitating adverse physical effects that could be of a serious or life-threatening nature as an unintended side effect of the pharmaceutical composition.

Opioid agonist analgesics have long been a cornerstone of pharmaceutical management of pain and other medical maladies such as cough, loose stool or diarrhea. However, use of opioid agonist analgesics may be accompanied by feeling euphoria as a reaction apart from relief of pain, or may be accompanied by other pharmaceutical effects as to create a wanting of the opioid agonist analgesic as an issue separate and distinct from the issue of pain relief. It is undesirable for a human patient to want to be administered an opioid agonist analgesic for reasons other than relief of pain or prescribed treatment of licit medical maladies such as loose stool. Such a wanting could result in the opioid agonist analgesic being administered in quantities greater than that required to treat pain and other licit medical maladies, which would result in waste of opioid agonist analgesic, and an increase in spending for opioid agonist analgesics. This is of great societal significance in managing the allocation of scarce resources available in the treating health care system in general. Any wastage of money on a pharmaceutical or medication results in less money available for other needed resources, be they other medications or health care services. In and of itself, a decrease in wanting of opioids apart from pain relief and other licit uses (hereafter “any licit use”) would be of great of great utility, whether it be in an opioid naïve individual (i.e., one that has not been previously exposed to opioid analgesics) or an individually chronically exposed to opioid agonist analgesics (e.g., a chronic pain patient, as one who is long suffering from malignant or cancer-related pain).

There have been attempts to reduce the effective amount of opioid agonist analgesic for any licit use. Such attempts have included the co-administration of opioids with NMDA-receptor antagonists or relatively low doses of opioid antagonist. These methods, if effective, could theoretically serve the desired purpose of reducing wastage of opioids, however these methods have not been demonstrated to decrease the wanting of the opioid apart from any licit use, and in fact, could theoretically potentiate the opioid agonist effect to possibly increase the wanting desire of the opioid agonist analgesic, which would have the opposite of the desired effect to decrease wastage and optimize management of scare health care resources.

Mayer, et al teach that NMDA (N-methyl-D-aspartate) receptor antagonists such as dextromethorphan or dextrophan may be combined with opioid agonist analgesics for the prevention of opioid tolerance (U.S. Pat. No. 5,654,281). However, this may make the opioid agonist effectively more potent, and Mayer does not teach that this invention will decrease the wanting or desire for being administered opioids apart from the effect of any licit use.

Caruso teaches that NMDA receptor antagonists administered with narcotic agonist/antagonists increase the analgesic effect of the agonist/antagonist (U.S. Pat. No. 6,007,841). Again, this may render the opioid agonist more potent and does not speak to decreasing the wanting of the opioid apart from the effect of any licit use. Caruso makes no mention of α3β4 nicotinic receptors or its antagonists.

Crain et al teach that very small doses of opioid antagonists (inverse opioid agonists) may potentiate the analgesic effect of opioid agonist analgesics (U.S. Pat. Nos. 5,580,876 and 5,767,125). Crain does not teach decreasing the wanting desire of opioid analgesics apart from any licit use, nor decreasing the tendency for illicit use. In fact, the technology taught by Crain et al, because it teaches potentiation of opioid analgesic effects by combining the analgesic with an inverse opioid agonist, may actually increase the tendency for illicit self-administration by a physically dependent human subject in direct contradistinction to the present invention.

The present author teaches that a pharmaceutical composition comprising nalmefene and an opioid agonist analgesic may optimize dopamine homeostasis while dissuading a human from abusing the opioid agonist analgesic (U.S. Pat. No. 6,103,258, hereafter “'258”). This invention, however, does not utilize nicotinic receptors, and it has a ceiling effect for any given combination of nalmefene and opioid. Further, specific drug combinations in varying ratios of nalmefene to opioid must be formulated to effectively deliver therapeutic doses of a particular opioid agonist analgesic.

Palermo, et al (U.S. Pat. No. 6,228,863) and Kaiko, et al (U.S. Pat. No. 6,277,384) teaching compositions for oral administration containing opioid agonist analgesics and opioid antagonists in varying amounts depending upon the particular opioid agonist and antagonist used. These formulations, however, have the potential to produce precipitated abstinence syndrome in susceptible individuals, unlike the present invention. Unintentional precipitated abstinence syndrome (“withdrawal”) can have serious deleterious effects on humans, such as precipitation of catecholamine release, exaggerated stress response and myocardial ischemia Unmonitored, as may occur with an unintentional withdrawal, this could be life threatening.

Smith, et al teach that a kappa-2 opioid receptor agonist may be combined with a mu opioid receptor agonist such that relatively low sub-therapeutic doses of each may result in therapeutic analgesia (U.S. Pat. No. 6,310,072). However, Smith does not teach that this invention reduces wanting for the combined drug combination apart from any licit use.

Kaiko and Colucci (U.S. Pat. No. 6,475,494 or “'494”) teach the combination of an opioid agonist analgesic and an inverse opioid agonist. They do not teach or claim the combination of an opioid agonist analgesic and a neutral receptor binding agent as in the present invention. The invention of '494 teaches an aversive reaction in physically dependent human subjects that the present invention modifies so as not to be so inhumane or dangerous to such physically dependent human subjects. Another important advantage of the present invention over '494 is that '494 includes the inverse opioid agonist naltrexone, which is metabolized in humans to 6-beta-naltrexol. Thus, with '494 the inverse opioid agonist naltrexone competes for binding to mu-opioid receptors with the neutral receptor binding agent 6-beta-naltrexol, complicating the predictability of the intended effect of the naltrexone, and reducing if not eliminating the beneficial effect of the 6-beta-naltrexol that may be present as a metabolite, as compared to naltrexone. '494 does not teach, contemplate or even hint at administering a neutral receptor binding agent or a partial mu-opioid agonist of the requisite efficacy and potency with an opioid agonist analgesic as described herein.

U.S. Patent Application No. 20010049375 of Sadee, et al. teach the administration of a neutral receptor binding agent solely as a method for the treatment of drug dependency. Sadee, et al. do not teach toward a method or pharmaceutical composition containing an opioid agonist analgesic and a neutral receptor binding agent at all, let alone as a means to administer an opioid agonist analgesic for any licit use to non-dependent humans with the objective(s) of the present invention.

### DETAILED DESCRIPTION OF THE INVENTION

The present invention combines an opioid analgesic with a compound, molecule or peptide that also binds an opioid receptor such that the compound, molecule or peptide will compete with the opioid analgesic for a binding site on the opioid receptor, where the compound, molecule or peptide has relatively low or negligible efficacy in comparison to the opioid analgesic, but which at a minimum will allow for the opioid receptor's intrinsic mu-receptor activity when bound to it.

An opioid receptor is acted upon by a compound, molecule or peptide (hereafter, “molecule”) in such a way that the molecule causes a change in physical conformation of the receptor such that the conformational change induces a concomitant change in an opioid receptor-linked protein (e.g., a “G-protein”) that is associated with induction of further chemical changes such as phosphorylation involving a protein or enzyme, or activation/inactivation of an enzyme such as adenyl cyclase, which further induces other chemical changes such as increase or reduction of “second messengers” such as adenosine-3′:5′-cyclic phosphate or “cAMP” (from interaction of adenosine monophosphate or “AMP” and ATP, e.g.). The molecule may bind the receptor with a relatively high affinity and a relatively high efficacy, such as the prototypical mu-opioid agonist analgesic. Alternatively, an opioid receptor may be left in absence of binding molecules, such that the second messengers are produced in reliance upon the intrinsic mu-receptor activity.

The conformational status of the receptor or the coupling of the opioid receptor-linked protein or the number of receptors available on the cell membrane, may be a function of the past history of exposure to opioid agonists and/or opioid antagonists (see Yoburn, et al. in Pharmacology Biochemistry and Behavior, Vol. 51, Nos. 2/3, pp. 535-539, 1995 and Paronis and Holtzman in Journal of Pharmacology and Experimental Therapeutics, Vol. 259, No. 2, pp. 582-9, 1991 and Liu and Prather in Molecular Pharmacology, Vol. 60, No. 1, pp. 53-62, 2001). Thus, intrinsic mu-receptor activity of an opioid receptor may be expected to differ depending upon whether the receptor is found in an opioid naïve individual, an opioid dependent or addicted individual, or an individual that is prescribed opioid antagonists (inverse opioid agonists).

An opioid dependent human may have a violent aversive reaction consistent with the phenomenon of acute withdrawal when subjected to an opioid antagonist (inverse opioid agonist), whereas an opioid naïve human may have no such reaction at all when subjected to the same amount and dosage of the inverse opioid agonist. The opioid dependent human just written about may experience much less an aversive reaction when administered a neutral receptor binding agent of equivalent potency to the opioid antagonist described in this paragraph, such a difference having great clinical significance. Acute opioid withdrawal of the kind being discussed is expected to increase firing of neurons in the locus coeruleus of the brain and be associated with clinically significant rises in catecholamines such as epinephrine, norepinephrine and dopamine. This surge in catecholamines increases the work load of the heart, and in a patient with compromised cardiovascular function can result in myocardial ischemia, irregular conduction of electrical currents throughout the heart, and other maladies that can cause great physical peril to the human experiencing such medical signs. Drug addicts are at increased risk for cardiac pathology involving conduction disturbances and blood supply to the heart, as well as valvular heart disease that can further complicate an aversive withdrawal reaction. Therefore, it is undesirable to administer a drug dosage form that is likely to, or intended to, cause such an aversive reaction in a human population expected to be administered opioid agonist analgesics.

As described by Wang, et al. (Journal of Neurochemistry, Vol. 77, No. 6, pp. 1590-1600, 2001) naltrexone is an inverse opioid agonist in animals pretreated with morphine. This model would indicate that humans physically dependent on opioid analgesics would respond to naltrexone as an inverse opioid agonist. However, 6-beta-naltrexol in animals acts as a neutral receptor binding agent irrespective of morphine pretreatment. It follows then that 6beta-naltexol would be a neutral receptor binding agent in humans dependent on or addicted to opioid analgesics.

Combinations of opioid agonist analgesics and the neutral receptor binding agent 6-beta-naltrexol which are orally administered in ratios which are equivalent to the ratio of, e.g., 6-beta-naltrexol to hydrocodone set forth herein are considered to be within the scope of the present invention. Equipotent doses of other neutral receptor binding agents such as 6-alpha-naltrexol, 6-alpha-naloxol, 6-beta-naloxol and 6-beta-naltrexamine in clinically equivalent ratios as set forth herein are also within the scope of the present invention.

It is known that when co-administered with morphine, heroin or other opioids on a chronic basis, naltrexone blocks the development of physical dependence to opioids. It is believed that the method by which naltrexone blocks the effects of heroin is by competitively binding at the opioid receptors. 6-alpha- and 6-beta-naltrexol and -naloxol, and 6beta-naltrexamine would therefore be expected to also prevent or reduce the development of tolerance, but with the previously mentioned great advantages of neutral receptor binding agents.

For example, in formulations of the present invention in which the opioid is hydrocodone bitartrate 15 mg, the amount of 6-beta-naltrexol included in the formulation is from about 0.5 mg to about 12 mg, and preferably from about 0.75 mg to about 8 mg 6-beta-naltrexol per 15 mg hydrocodone. The ratio described herein is based on the following calculations from Kaiko and Colucci ('494), and extrapolating from data from Rukstalis, et al. (Alcoholism Clinical and Experimental Research, Vol. 24, No. 10, pp. 1593-96, October 2000) in which 2.6 times the dose of naltrexone was used for 6-beta-naltrexol in treating Wistar rats:

Equianalgesic Doses of Opioids

| Opioid | Calculated Dose (mg) |
|---|---|
| Oxycodone | 13.5 |
| Codeine | 90.0 |
| Hydrocodone | 15.0 |
| Hydromorphone | 3.375 |
| Levorphanol | 1.8 |
| Meperidine | 135.0 |
| Methadone | 9.0 |
| Morphine | 27.0 |

Based on a preferred ratio of 6-beta-naltexol per 15 mg of hydrocodone, the approximate ratio of 6-beta-naltrexol to 1 mg of each opioid is set forth below:

Weight Ratio of 6-beta-naltrexone per Dose Opioid

| | Weight Ratio 6-beta-naltrexol per |
|---|---|
| Opioid Agonist | 1 mg Opioid Agonist |
| Oxycodone | 0.020 to 0.770 of 6-beta-naltrexol |
| Codeine | 0.003 to 0.114 of 6-beta-naltrexol |
| Hydrocodone | 0.018 to 0.694 of 6-beta-naltrexol |
| Hydromorphone | 0.089 to 3.082 of 6-beta-naltrexol |
| Levorphanol | 0.164 to 5.777 of 6-beta-naltrexol |
| Meperidine | 0.002 to 0.077 of 6-beta-naltrexol |
| Methadone | 0.031 to 1.154 of 6-beta-naltrexol |
| Morphine | 0.010 to 0.385 of 6-beta-naltrexol |

Of course, there may be differences in both efficacy and potency of 6-beta-naltrexol among the Wistar rats and humans, and a more definitive ratio of neutral binding agent to opioid analgesic should ideally be calculated. However, in light of the present invention, one skilled in the art would be able to do so in routine laboratory trials prior to large scale clinical testing as is typically carried out in the United States pharmaceutical industry. In fact, Kaiko and Colucci lay the foundation for such routine testing in '494. For example, The amount of neutral binding agent which is useful to achieve an optimal ratio of opioid agonist to neutral receptor binding agent may be determined at least in part, for example, through the use of “surrogate” tests, such as a VAS scale (where the subject grades his/her perception of the effect of the dosage form) and/or via a measurement such as pupil size (measured by pupillometry). Other commonly employed instruments in the industry are the Addiction Research Center Inventory (“ARCI”) and the POMS rating scale. In addiction, a technique such as applying a low amp electrical current transcutaneously to a subject and recording the amperage at various stages of discomfort is a way to determine or simulate effective analgesia. These techniques are well described in the literature and are well known to one skilled in the art.

Such measurements allow one skilled in the art to determine the dose of neutral receptor binding agent relative to the dose of opioid agonist analgesic that causes a diminution in the opioid effects of the agonist. Subsequently, one skilled in the art can determine the level of neutral receptor binding agent that causes a reduction in the slope of the dose-response curve for the opioid as well as the level of neutral receptor binding agent that minimizes “liking scores” or opioid reinforcing properties in human subjects.

Other factors in extrapolating dosages of 6-beta-naltrexol from dosages of naltrexone are the relative biological and elimination half-lives of the respective drugs, the volumes of distribution of the respective drugs, and the bioavailability according to the route of administration, i.e., “is the bioavailabilty of one drug similar or different from that of the other drug?” However, these techniques are industry standards and are well known to one skilled in the art, hence in light of the present invention the optimal ratio of neutral receptor binding agent to opioid agonist analgesic is readily obtained. Incorporated herein by way of reference are Ferrari, et al. “Serum time course of naltrexone and 6-beta-naltrexol levels during long-term treatment in drug addicts,” (Drug and Alcohol Dependence, Vol. 52, No. 3, pp. 211-220, 1998), and Porter, et al., “In vivo and in vitro potency studies of 6-beta-naltrexol, the major human metabolite of naltrexone,” (Addiction Biology, Vol. 7, No. 2, pp. 219-225, April 2002).

Opioid analgesics which are useful in the present invention include all opioid agonist analgesics that have a greater efficacy than the particular neutral receptor binding agent or partial mu-opioid agonist of the invention, including but not limited to alfentanil, allylprodine, alphaprodine, anileridine, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, fentanyl, heroin, hydrocodone, hydromorphone, hydroxypethidine, isomethadone, ketobemidone, levorphanol, levophcnacylmorphan, lofentanil, meperidine, meptazinol, metazocine, methadone, metopon, morphine, myrophine, narceine, nicomorphine, norlevorphanol, normethadone, noroxycodone, nalorphine, nalbuphene, normorphine, norpipanone, opium, oxycodone, oxymorphone, papaveretum, pentazocine, phenadoxone, phenomorphan, phenazocine, phenoperidine, piminodine, piritramide, propheptazine, promedol, properidine, pro-poxyphene, sufentanil, tilidine, tramadol, mixtures of any of the foregoing, salts of any of the foregoing, and the like.

In certain preferred embodiments, the opioid agonist analgesic is selected from the group consisting of hydrocodone, morphine, hydromorphone, oxycodone, codeine, levorphanol, meperidine, methadone, or salts thereof, or mixtures thereof. In certain preferred embodiments, the opioid agonist is hydrocodone. Equianalgesic doses of these opioids, in comparision to a 15 mg dose of hydrocodone, are set forth herein (above).

Although hydrocodone is effective in the management of pain, there has been an increase in its abuse by individuals who are psychologically dependent on opioids or who misuse opioids for illicit reasons. Previous experience with other opioids has demonstrated a decreased abuse potential when opioids are administered in combination with a narcotic antagonist especially in patients who are ex-addicts. Weinhold L L, et al. “Buprenorphine Alone and in Combination with Naltrexone in Non-Dependent Humans” (Drug and Alcohol Dependence 1992; 30:263-274); Mendelson J., et. al., “Buprenorphine and Naloxone Interactions in Opiate-Dependent Volunteers,” (Clin Pharm Ther 1996; 60:105-114); both of which are hereby incorporated by reference.

Hydrocodone is a semisynthetic narcotic analgesic and antitussive with multiple central nervous system and gastrointestinal actions. Chemically, hydrocodone is 4,5-epoxy-3-methoxy-17-methylmorphinan-6-one, and is also known as dihydrocodeinone. Like other opioids, hydrocodone may be habit forming and may produce drug dependence of the morphine type. In excess doses hydrocodone, like other opium derivatives, will depress respiration.

Oral hydrocodone is also available in Europe (Belgium, Germany, Greece, Italy, Luxembourg, Norway and Switzerland) as an antitussive agent. A parenteral formulation is also available in Germany as an antitussive agent. For use as an analgesic, hydrocodone bitartrate is commercially available in the United States only as a fixed combination with non-opiate drugs (i.e., acetaminophen, aspirin, ibuprofen, etc.) for relief of moderate or moderately severe pain. Administration of available hydrocodone preparations in excess of prescribed dosing can therefore lead to ingested of supra-therapeutic doses of acetaminophen or NSAID, possibly leading to toxic physical reactions such liver toxicity or gastrointestinal hemorrhage. Therefore, it would be of great utility to decrease the tendency toward excessive administration or ingestion of available hydrocodone pharmaceuticals.

A common dosage form of hydrocodone is in combination with acetaminophen, and is commercially available, e.g., as Lortab® in the U.S. from UCB Pharma, Inc. as 2.5/500 mg, 5/500 mg, 7.5/500 mg and 10/500 mg hydrocodone/acetaminophen tablets. Tablets are also available in the ratio of 7.5 mg hydrocodone bitartrate and 650 mg acetaminophen; and 7.5 mg hydrocodone bitartrate and 750 mg acetaminophen. Hydrocodone in combination with aspirin is given in an oral dosage form to adults generally in 1-2 tablets every 4-6 hours as needed to alleviate pain. The tablet form is 5 mg hydrocodone bitartrate and 224 mg aspirin with 32 mg caffeine; or 5 mg hydrocodone bitartrate and 500 mg aspirin. A relatively new formulation comprises hydrocodone bitartrate and ibuprofen. Vicoprofen®, commercially available in the U.S. from Knoll Laboratories, is a tablet containing 7.5 mg hydrocodone bitartrate and 200 mg ibuprofen. The present invention is contemplated to encompass all such formulations, with the inclusion of any orally active neutral receptor binding agents or partial-mu opioid agonists with potency and efficacy profiles within the inventive parameters set forth herein.

The abuse potential of opioid analgesics such as hydrocodone is curtailed by the inventive combinations of the present invention. More particularly, it has been discovered that it is possible to combine in a single oral dosage form an opioid analgesic together with a small amount of neutral receptor binding agent or partial mu-opioid agonist, to achieve a product which still provides analgesia but which diminishes the likelihood that a human subject will abuse the drug by taking more than one tablet at a time, e.g., 2-3 times more than the usually prescribed dose.

The combination of opioid agonist analgesic and neutral receptor binding agent or partial mu-opioid agonist can be employed in admixtures with conventional excipients, i.e., pharmaceutically acceptable organic or inorganic carrier substances suitable for oral administration, known to the art. Suitable pharmaceutically acceptable carriers include but are not limited to water, salt solutions, alcohols, gum arabic, vegetable oils, benzyl alcohols, polyethylene glycols, gelate, carbohydrates such as lactose, amylose or starch, magnesium stearate talc, silicic acid, viscous paraffin, perfume oil, fatty acid monoglycerides and diglycerides, pentaerythritol fatty acid esters, hydroxymethylcellulose, polyvinylpyrrolidone, etc. The pharmaceutical preparations can be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They can also be combined where desired with other active agents, e.g., other analgesic agents. For oral administration, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients which are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay release of the active ingredients. Formulations for oral use may also be presented as hard gelatin capsules wherein the active ingredient is mixed with an inert diluent.

Aqueous suspensions contain the above-identified combination of drugs and that mixture has one or more excipients suitable as suspending agents, for example pharmaceutically acceptable synthetic gums such as hydroxypropylmethylcellulose or natural gums. Oily suspensions may be formulated by suspending the above-identified combination of drugs in a vegetable oil or mineral oil. The oily suspensions may contain a thickening agent such as beeswax or cetyl alcohol. A syrup, elixir, or the like can be used wherein a sweetened vehicle is employed. Injectable suspensions may also be prepared, in which case appropriate liquid carriers, suspending agents and the like may be employed.

The method of treatment and pharmaceutical formulations of the present invention may further include one or more drugs in addition to the opioid analgesic and neutral receptor binding agent or partial mu-opioid agonist, which additional drug(s) may or may not act synergistically therewith. Thus, in certain embodiments, a combination of two opioid analgesics may be included in the formulation, in addition to a neutral receptor binding agent. For example, the, dosage form may include two opioid analgesics having different properties, such as half-life, solubility, potency, and a combination of any of the foregoing. In yet further embodiments, one or more opioid analgesics is included and a further non-opioid drug is also included, in addition to the neutral receptor binding agent or partial mu-opioid agoinist. Such non-opioid drugs would preferably provide additional analgesia, and include, for example, aspirin; acetaminophen; non-sterioidal antiinflammatory drugs (“NSAIDS”), e.g., ibuprofen, ketoprofen, etc.; N-methyl-D-aspartate (NMDA) receptor antagonists, e.g., a morphinan such as dextromethorphan or dextrorphan, or ketamine or d-methadone; cycooxygenase-II inhibitors (“COX-II inhibitors”); glycine receptor antagonists; and/or alpha3-beta4 nicotinic receptor antagonists.

In certain preferred embodiments of the present invention, the invention allows for the use of lower doses of the opioid analgesic by virtue of the inclusion of an additional non-opioid agonist, such as an NSAID or a COX-2 inhibitor. By using lower amounts of either or both drugs, the side effects associated with effective pain management in humans are reduced.

Suitable non-steroidal anti-inflammatory agents, including ibuprofen, diclofenac, naproxen, benoxaprofen, flurbiprofen, fenoprofen, flubufen, ketoprofen, indoprofen, piroprofen, carprofen, oxaprozin, pramoprofen, muroprofen, trioxaprofen, suprofen, aminoprofen, tiaprofenic acid, fluprofen, bucloxic acid, indomethacin, sulindac, tolmetin, zomepirac, tiopinac, zido-metacin, acemetacin, fentiazac, clidanac, oxpinac, mefenamic acid, meclofenamic acid, flufenamic acid, niflumic acid, tolfenamic acid, diflurisal, flufenisal, piroxicam, sudoxicam or isoxicam, and the like. Useful dosages of these drugs are well known to those skilled in the art.

N-methyl-D-aspartate (NMDA) receptor antagonists are well known in the art, and encompass, for example, morphinans such as dextromethorphan or dextrorphan, ketamine, d-methadone or pharmaceutically acceptable salts thereof. For purposes of the present invention, the term “NMDA antagonist” is also deemed to encompass drugs that block a major intracellular consequence of NMDA-receptor activation, e.g. a ganglioside such as GM₁ or GT_{1b} a phenothiazine such as trifluoperazine or a naphthalenesulfonamide such as N-(6-aminothexyl)-5-chloro-1-naphthalenesulfonamide. These drugs are stated to inhibit the development of tolerance to and/or dependence on addictive drugs, e.g., narcotic analgesics such as morphine, codeine, etc. in U.S. Pat. Nos. 5,321,012 and 5,556,838 (both to Mayer, et. al.), and to treat chronic pain in U.S. Pat. No. 5,502,058 (Mayer, et. al.), all of which are hereby incorporated by reference. The NMDA antagonist may be included alone, or in combination with a local anesthetic such as lidocaine, as described in these Mayer, et al. patents.

The treatment of chronic pain via the use of glycine receptor antagonists and the identification of such drugs is described in U.S. Pat. No. 5,514,680 (Weber, et al.), hereby incorporated by reference.

COX-2 inhibitors have been reported in the art and many chemical structures are known to produce inhibition of cyclooxygenase-2. COX-2 inhibitors are described, for example, in U.S. Pat. Nos. 5,616,601; 5,604,260; 5,593,994; 5,550,142; 5,536,752; 5,521,213; 5,475,995; 5,639,780; 5,604,253; 5,552,422; 5,510,368; 5,436,265; 5,409,944; and 5,130,311, all of which are hereby incorporated by reference. Certain preferred COX-2 inhibitors include celecoxib (SC-58635), DUP-697, flosulide (CGP-28238), meloxicam, 6-methoxy-2 naphthylacetic acid (6-MNA), MK-966, nabumetone (prodrug for 6-MNA), nimesulide, NS-398, SC-5766, SC-58215, T-614; or combinations thereof Dosage levels of COX-2 inhibitor on the order of from about 0.005 mg to about 140 mg per kilogram of body weight per day are therapeutically effective in combination with an opioid analgesic. Alternatively, about 0.25 mg to about 7 g per patient per day of a COX-2 inhibitor is administered in combination with an opioid analgesic.

In yet further embodiments, a non-opioid drug can be included which provides a desired effect other than analgesia, e.g., antitussive, expectorant, decongestant, antihistamine drugs, local anesthetics, and the like.

In another preferred embodiment, a nicotinic receptor antagonist can be included, most preferably an alpha-3-beta-4-nicotinic receptor antagonist as described by the present inventor in U.S. patent application Ser. No. 10/127,358 which is hereby incorporated by reference.

An oral dosage form according to the invention may be provided as, for example, granules, spheroids, beads, pellets (hereinafter collectively referred to as “multiparticulates”). An amount of the multiparticulates which is effective to provide the desired dose of opioid over time may be placed in a capsule or may be incorporated in any other suitable oral solid form. Alternatively, the oral dosage form may be in the form of a tablet.

Controlled Release Dosage Forms

The opioid agonist/neutral receptor binding agent combination can be formulated as a controlled or sustained release oral formulation in any suitable tablet, coated tablet or multiparticulate formulation known to those skilled in the art. The sustained release dosage form may optionally include a sustained release carrier that is incorporated into a matrix along with the opioid agonist and opioid antagonist, or may be applied as a sustained release coating.

In embodiments in which the opioid analgesic comprises hydrocodone, the sustained release oral dosage forms may include analgesic doses from about 8 mg to about 50 mg of hydrocodone per dosage unit. In sustained release oral dosage forms where hydromorphone is the therapeutically active opioid, it is included in an amount from about 2 mg to about 64 mg hydromorphone hydrochloride. In another embodiment, the opioid analgesic comprises morphine, and the sustained release oral dosage forms of the present invention include from about 2.5 mg to about 800 mg morphine, by weight. In yet another embodiment, the opioid analgesic comprises oxycodone and the sustained release oral dosage forms include from about 2.5 mg to about 800 mg oxycodone. The opioid analgesic may comprise tramadol and the sustained release oral dosage forms may include from about 25 mg to 800 mg tramadol per dosage unit. The dosage form may contain more than one opioid analgesic to provide a substantially equivalent therapeutic effect. Alternatively, the dosage form may contain molar equivalent amounts of other salts of the opioids useful in the present invention.

In one preferred embodiment of the present invention, the sustained release dosage form comprises such particles containing or comprising the active ingredient, wherein the particles have diameter from about 0.1 mm to about 2.5 mm, preferably from about 0.5 mm to about 2 mm.

The particles are preferably film coated with a material that permits release of the opioid agonist/neutral receptor binding agent combination at a sustained rate in an aqueous medium. The film coat is chosen so as to achieve, in combination with the other stated properties, a desired in-vitro release rate. The sustained release coating formulations of the present invention should be capable of producing a strong, continuous film that is smooth and elegant, capable of supporting pigments and other coating additives, non-toxic, inert, and tack-free.

In certain embodiments, the particles comprise normal release matrixes containing the opioid analgesic with the neutral receptor binding agent or partial mu-opioid agonist.

Coatings

The dosage forms of the present invention may optionally be coated with one or more materials suitable for the regulation of release or for the protection of the formulation. In one embodiment, coatings are provided to permit either pH-dependent or pH-independent release, e.g., when exposed to gastrointestinal fluid. A pH-dependent coating serves to release the opioid in desired areas of the gastro-intestinal (“GI”) tract, e.g., the stomach or small intestine, such that an absorption profile is provided which is capable of providing at least about eight hours and preferably about twelve hours to up to about twenty-four hours of analgesia to a patient. When a pH-independent coating is desired, the coating is designed to achieve optimal release regardless of pH-changes in the environmental fluid, e.g., the GI tract. It is also possible to formulate compositions that release a portion of the dose in one desired area of the GI tract, e.g., the stomach, and release the remainder of the dose in another area of the GI tract, e.g., the small intestine.

Formulations according to the invention that utilize pH-dependent coatings to obtain formulations may also impart a repeat-action effect whereby unprotected drug is coated over the enteric coat and is released in the stomach, while the remainder, being protected by the enteric coating, is released further down the gastrointestinal tract. Coatings which are pH-dependent may be used in accordance with the present invention include shellac, cellulose acetate phthalate (CAP), polyvinyl acetate phthalate PVAP), hydroxypropylmethylcellulose phthalate, and methacrylic acid ester copolymers, zein, and the like.

In certain preferred embodiments, the substrate (e.g., tablet core bead, matrix particle) containing the opioid analgesic (with or without the COX-2 inhibitor) is coated with a hydrophobic material selected from (i) an alkylcellulose; (ii) an acrylic polymer; or (iii) mixtures thereof. The coating may be applied in the form of an organic or aqueous solution or dispersion. The coating may be applied to obtain a weight gain from about 2 to about 25% of the substrate in order to obtain a desired sustained release profile. Coatings derived from aqueous dispersions—are described, e.g., in detail in U.S. Pat. Nos. 5,273,760 and 5,286,493, hereby incorporated by reference.

Other examples of sustained release formulations and coatings which may be used in accordance with the present invention include U.S. Pat. Nos. 5,324,351; 5,356,467, and 5,472,712, hereby incorporated by reference in their entirety.

Alkycellulose Polymers

Cellulosic materials and polymers, including alkylcelluloses, provide hydrophobic materials well suited for coating the beads according to the invention. Simply by way of example, one preferred alkylcellulosic polymer is ethylcellulose, although the artisan will appreciate that other cellulose and/or alkylcellulose polymers may be readily employed, singly or in any combination, as all or part of a hydrophobic coating according to the invention.

One commercially available aqueous dispersion of ethylcellulose is Aquacoat® (FMC Corp., Philadelphia, Pa., U.S.A.). Aquacoat® is prepared by dissolving the ethylcellulose in a water-immiscible organic solvent and then emulsifying the same in water in the presence of a surfactant and a stabilizer. After homogenization to generate submicron droplets, the organic solvent is evaporated under vacuum to form a pseudolatex. The plasticizer is not incorporated in the pseudolatex during the manufacturing phase. Thus, prior to using the same as a coating, it is necessary to intimately mix the Aquacoat® with a suitable plasticizer prior to use.

Another aqueous dispersion of ethylcellulose is commercially available as Surelease® (Colorcon, Inc., West Point, Pa., U.S.A.). This product is prepared by incorporating plasticizer into the dispersion during the manufacturing process. A hot melt of a polymer, plasticizer (dibutyl sebacate), and stabilizer (oleic acid) is prepared as a homogeneous mixture, which is then diluted with an alkaline solution to obtain an aqueous dispersion which can be applied directly onto substrates.

Acrylic Polymers

In other preferred embodiments of the present invention, the hydrophobic material comprising the controlled release coating is a pharmaceutically acceptable acrylic polymer, including but not limited to acrylic acid and methacrylic acid copolymers, methyl methacrylate copolymers, ethoxyethyl methacrylates, cyanoethyl methacrylate, poly(acrylic acid), poly(methacrylic acid), methacrylic acid alkylamide copolymer, poly(methyl methacrylate), polymethacrylate, poly(methyl methacrylate) copolymer, polyacrylamide, aminoalkyl methacrylate copolymer, poly(methacrylic acid anhydride), and glycidyl methacrylate co-polymers.

In certain preferred embodiments, the acrylic polymer is comprised of one or more ammonio methacrylate copolymers. Ammonio methacrylate copolymers are well known in the art, and are described in NF XVII as fully polymerized copolymers of acrylic and methacrylic acid esters with a low content of quaternary ammonium groups.

In order to obtain a desirable dissolution profile, it may be necessary to incorporate two or more ammonio methacrylate copolymers having differing physical properties, such as different molar ratios of the quaternary ammonium groups to the neutral (meth)acrylic esters.

Certain methacrylic acid ester-type polymers are useful for preparing pH-dependent coatings which may be used in accordance with the present invention. For example, there are a family of copolymers synthesized from diethylaminoethyl methacrylate and other neutral methacrylic esters, also known as methacrylic acid copolymer or polymeric methacrylates, commercially available as Eudragite® from Rohm Tech, Inc. There are several different types of Eudragit®. For example, Eudragit® E is an example of a methacrylic acid copolymer which swells and dissolves in acidic media. Eudragit® L is a methacrylic acid copolymer which does not swell at about pH<5.7 and is soluble at about pH>6. Eudragit® RS does not swell at about pH<6.5 and is soluble at about pH>7. Eudragit® RL and Eudragit® RS are water swellable, and the amount of water absorbed by these polymers is pH-dependent, however, dosage forms coated with Eudragit® RL and RS are pH-independent.

In certain preferred embodiments, the acrylic coating comprises a mixture of two acrylic resin lacquers commercially available from Rohm Pharma under the Tradenames Eudragie® RL30D and Eudragit® RS30D, respectively. Eudragit® RL30D and Eudragit® RS30D are copolymers of acrylic and methacrylic esters with a low content of quaternary ammonium groups, the molar ratio of ammonium groups to the remaining neutral (meth)acrylic esters being 1:20 in Eudragit® RL30D and 1:40 in Eudragit® RS30D. The mean molecular weight is about 150,000. The code designations RL (high permeability) and RS (low permeability) refer to the permeability properties of these agents. Eudragit® RL/RS mixtures are insoluble in water and in digestive fluids. However, coatings formed from the same are swellable and permeable in aqueous solutions and digestive fluids.

The Eudragit® RL/RS dispersions of the present invention may be mixed together in any desired ratio in order to ultimately obtain a sustained release formulation having a desirable dissolution profile. Desirable sustained release formulations may be obtained, for instance, from a retardant coating derived from 100% Eudragit® RL, 50% Eudragit® RL and 50% Eudragit® RS, and 10% Eudragit® RL:Eudragit® 90% RS. Of course, one skilled in the art will recognize that other acrylic polymers may also be used, such as, for example, Eudragit® L.

Plasticizers

In embodiments of the present invention where the coating comprises an aqueous dispersion of a hydrophobic material, the inclusion of an effective amount of a plasticizer in the aqueous dispersion of hydrophobic material will further improve the physical properties of the sustained release coating. For example, because ethylcellulose has a relatively high glass transition temperature and does not form flexible films under normal coating conditions, it is preferable to incorporate a plasticizer into an ethylcellulose coating containing sustained release coating before using the same as a coating material. Generally, the amount of plasticizer included in a coating solution is based on the concentration of the film-former, e.g., most often from about 1 to about 50 percent by weight of the film-former. Concentration of the plasticizer, 30 however, can only be properly determined after careful experimentation with the particular coating solution and method of application.

Examples of suitable plasticizers for ethylcellulose include water insoluble plasticizers such as dibutyl sebacate, diethyl phthalate, triethyl citrate, tributyl citrate, and triacetin, although it is possible that other water-insoluble plasticizers (such as acetylated monoglycerides, phthalate esters, castor oil, etc.) may be used. Triethyl citrate is an especially preferred plasticizer for the aqueous dispersions of ethyl cellulose of the present invention.

Examples of suitable plasticizers for the acrylic polymers of the present invention include, but are not limited to citric acid esters such as triethyl citrate NF XVI, tributyl citrate, dibutyl phthalate, and possibly 1,2-propylene glycol. Other plasticizers which have proved to be suitable for enhancing the elasticity of the films formed from acrylic films such as Eudragit® RL/RS lacquer solutions include polyethylene glycols, propylene glycol, diethyl phthalate, castor oil, and triacetin. Triethyl citrate is an especially preferred plasticizer for the aqueous dispersions of ethyl cellulose of the present invention.

It has further been found that the addition of a small amount of talc reduces the tendency of the aqueous dispersion to stick during processing, and acts as a polishing agent.

Processes for Preparing Coated Beads

When a hydrophobic material is used to coat inert pharmaceutical beads such as nu pariel 18/20 beads, a plurality of the resultant solid controlled release beads may thereafter be placed in a gelatin capsule in an amount sufficient to provide an effective controlled release dose when ingested and contacted by an environmental fluid, e.g., gastric fluid or dissolution media.

The controlled release bead formulations of the present invention slowly release the therapeutically active agent, e.g., when ingested and exposed to gastric fluids, and then to intestinal fluids. The controlled release profile of the formulations of the invention can be altered, for example, by varying the amount of overcoating with the hydrophobic material, altering the manner in which the plasticizer is added to the hydrophobic material, by varying the amount of plasticizer relative to hydrophobic material, by the inclusion of additional ingredients or excipients, by altering the method of manufacture, etc. The dissolution profile of the ultimate product may also be modified, for example, by increasing or decreasing the thickness of the retardant coating.

Spheroids or beads coated with a therapeutically active agent are prepared, e.g., by dissolving the therapeutically active agent in water and then spraying the solution onto a substrate, for example, nu pariel 18/20 beads, using a Wuster insert. Optionally, additional ingredients are also added prior to coating the beads in order to assist the binding of the opioid to the beads, and/or to color the solution, etc. For example, a product which includes hydroxypropylmethylcellulose, etc. with or without colorant (e.g., Opadry® commercially available from Colorcon, Inc.) may be added to the solution and the solution mixed (e.g., for about 1 hour) prior to application of the same onto the beads. The resultant coated substrate, in this example beads, may then be optionally overcoated with a barrier agent, to separate the therapeutically active agent from the hydrophobic controlled release coating. An example of a suitable barrier agent is one which comprises hydroxypropylmethylcellulose. However, any film-former known in the art may be used. It is preferred that the barrier agent does not affect the dissolution rate of the final product. The beads may then be overcoated with an aqueous dispersion of the hydrophobic material. The aqueous dispersion of hydrophobic material preferably further includes an effective amount of plasticizer, e.g. triethyl citrate. Pre-formulated aqueous dispersions of ethyl-cellulose, such as Aquacoat® or Surelease®, may be used. If Surelease® is used, it is not necessary to separately add a plasticizer. Alternatively, pre-formulated aqueous dispersions of acrylic polymers such as Eudragit® can be used.

The coating solutions of the present invention preferably contain, in addition to the film-former, plasticizer, and solvent system (i.e., water), a colorant to provide elegance and product distinction. Color may be added to the solution of the therapeutically active agent instead, or in addition to the aqueous dispersion of hydrophobic material. For example, color may be added to Aquacoat® via the use of alcohol or propylene glycol based color dispersions, milled aluminum lakes and opacifiers such as titanium dioxide by adding color with shear to water soluble polymer solution and then using low shear to the plasticized Aquacoat®. Alternatively, any suitable method of providing color to the formulations of the present invention may be used. Suitable ingredients for providing color to the formulation when an aqueous dispersion of an acrylic polymer is used include titanium dioxide and color pigments, such as iron oxide pigments. The incorporation of pigments, may, however, increase the retard effect of the coating.

Plasticized hydrophobic material may be applied onto the substrate comprising the therapeutically active agent by spraying using any suitable spray equipment known in the art. In a preferred method, a Wurster fluidized-bed system is used in which an air jet, injected from underneath, fluidizes the core material and effects, drying while the acrylic polymer coating is sprayed on. A sufficient amount of the hydrophobic material to obtain a predetermined controlled release of said therapeutically active agent when the coated substrate is exposed to aqueous solutions, e.g. gastric fluid, is preferably applied, taking into account the physical characteristics of the therapeutically active agent, the manner of incorporation of the plasticizer, etc. After coating with the hydrophobic material, a further overcoat of a film-former, such as Opadry® is optionally applied to the beads. This overcoat is provided, if at all, in order to substantially reduce agglomeration of the beads.

The release of the therapeutically active agent from the controlled release formulation of the present invention can be further influenced, i.e., adjusted to a desired rate, by the addition of one or more release-modifying agents, or by providing one or more passageways through the coating. The ratio of hydrophobic material to water soluble material is determined by, among other factors, the release rate required and the solubility characteristics of the materials selected.

The release-modifying agents which function as pore-formers may be organic or inorganic, and include materials that can be dissolved, extracted or leached from the coating in the environment of use. The pore-formers may comprise one or more hydrophilic materials such as hydroxypropylmethylcellulose.

The sustained release coatings of the present invention can also include erosion-promoting agents such as starch and gums.

The sustained release coatings of the present invention can also include materials useful for making microporous lamina in the environment of use, such as polycarbonates comprised of linear polyesters of carbonic acid in which carbonate groups reoccur in the polymer chain.

The release-modifying agent may also comprise a semi-permeable polymer.

In certain preferred embodiments, the release-modifying agent is selected from hydroxypropylmethylcellulose, lactose, metal stearates, and mixtures of any of the foregoing.

The sustained release coatings of the present invention may also include an exit means comprising at least one passageway, orifice, or the like. The passageway may be formed by such methods as those disclosed in U.S. Pat. Nos. 3,845,770; 3,916,889; 4,063,064; and 4,088,864 (all of which are hereby incorporated by reference). The passageway can have any shape such as round, triangular, square, elliptical, irregular, etc.

Matrix Bead Formulations

In other embodiments of the present invention, the controlled release formulation is achieved via a matrix having a controlled release coating as set forth above. The present invention may also utilize a controlled release matrix that affords in-vitro dissolution rates of the opioid within the preferred ranges and that releases the opioid in a pH-dependent or pH-independent manner. The materials suitable for inclusion in a controlled release matrix will depend on the method used to form the matrix. For example, a matrix in addition to the opioid analgesic and (optionally) COX-2 may include:

Hydrophilic and/or hydrophobic materials, such as gums, cellulose ethers, acrylic resins, protein derived materials; the list is not meant to be exclusive, and any pharmaceutically acceptable hydrophobic material or hydrophilic material which is capable of imparting controlled release of the active agent and which melts (or softens to the extent necessary to be extruded) may be used in accordance with the present invention.

Digestible, long chain (C.sub.8-C.sub.50, especially C.sub.12-C.sub.40), substituted or unsubstituted hydrocarbons, such as fatty acids, fatty alcohols, glyceryl esters of fatty acids, mineral and vegetable oils and waxes, and stearyl alcohol; and polyalkylene glycols. Of these polymers, acrylic polymers, especially Eudragit® RSPO—the cellulose ethers, especially hydroxyalkylcelluloses and carboxyalkylcelluloses, are preferred. The oral dosage form may contain between 1% and 80% (by weight) of at least one hydrophilic or hydrophobic material.

When the hydrophobic material is a hydrocarbon, the hydrocarbon preferably has a melting point of between 25.degree. and 90.degree. C. Of the long chain hydrocarbon materials, fatty (aliphatic) alcohols are preferred. The oral dosage form may contain up to 60% (by weight) of at least one digestible, long chain hydrocarbon.

Preferably, the oral dosage form contains up to 60% (by weight) of at least one polyalkylene glycol.

The hydrophobic material is preferably selected from the group consisting of alkylcelluloses, acrylic and methacrylic acid polymers and copolymers, shellac, zein, hydrogenated castor oil, hydrogenated vegetable oil, or mixtures thereof. In certain preferred embodiments of the present invention, the hydrophobic material is a pharmaceutically acceptable acrylic polymer, including but not limited to acrylic acid and methacrylic acid copolymers, methyl methacrylate, methyl methacrylate copolymers, ethoxyethyl methacrylates, cyanoethyl methacrylate, aminoalkyl methacrylate copolymer, poly(acrylic acid), poly(methacrylic acid), methacrylic acid allylamine copolymer, poly(methyl methacrylate), poly(methacrylic acid)(anhydride), polymethacrylate, polyacrylamide, poly(methacrylic acid anhydride), and glycidyl methacrylate copolymers. In other embodiments, the hydrophobic material is selected from materials such as hydroxyalkylcelluloses such as hydroxypropylmethylcellulose and mixtures of the foregoing.

Preferred hydrophobic materials are water-insoluble with more or less pronounced hydrophilic and/or hydrophobic trends. Preferably, the hydrophobic materials useful in the invention have a melting point from about 300 to about 200.degree. C., preferably from about 45.degree. to about 90.degree. C. Specifically, the hydrophobic material may comprise natural or synthetic waxes, fatty alcohols (such as lauryl, myristyl, stearyl, cetyl or preferably cetostearyl alcohol), fatty acids, including but not limited to fatty acid esters, fatty acid glycerides (mono-, di-, and tri-glycerides), hydrogenated fats, hydrocarbons, normal waxes, stearic aid, stearyl alcohol and hydrophobic and hydrophilic materials having hydrocarbon backbones. Suitable waxes include, for example, beeswax, glycowax, castor wax and carnauba wax. For purposes of the present invention, a wax-like substance is defined as any material which is normally solid at room temperature and has a melting point of from about 30.degree. to about 100.degree. C.

Suitable hydrophobic materials which may be used in accordance with the present invention include digestible, long chain (C₈-C₅₀, especially C₁₂-C₄₀), substituted or unsubstituted hydrocarbons, such as fatty acids, fatty alcohols, glyceryl esters of fatty acids, mineral and vegetable oils and natural and synthetic waxes. Hydrocarbons having a melting point of between 25 degrees and 90 degrees Celsius are preferred. Of the long chain hydrocarbon materials, fatty (aliphatic) alcohols are preferred in certain embodiments. The oral dosage form may contain up to 60% (by weight) of at least one digestible, long chain hydrocarbon.

Preferably, a combination of two or more hydrophobic materials are included in the matrix formulations. If an additional hydrophobic material is included, it is preferably selected from natural and synthetic waxes, fatty acids, fatty alcohols, and mixtures of the same. Examples include beeswax, carnauba wax, stearic acid and stearyl alcohol. This list is not meant to be exclusive.

One particular suitable matrix comprises at least one water soluble hydroxyalkyl cellulose, at least one C₁₂-C₃₆, preferably C₁₄-C₂₂, aliphatic alcohol and, optionally, at least one polyalkylene glycol. The at least one hydroxyalkyl cellulose is preferably a hydroxy (C₁ to C₆) alkyl cellulose, such as hydroxypropylcellulose, hydroxypropylmethylcellulose and, especially, hydroxyethylcellulose. The amount of the at least one hydroxyalkyl cellulose in the present oral dosage form will be determined, inter alia, by the precise rate of opioid release required. The at least one aliphatic alcohol may be, for example, lauryl alcohol, myristyl alcohol or stearyl alcohol. In particularly preferred embodiments of the present oral dosage form, however, the at least one aliphatic alcohol is acetyl alcohol or acetostearyl alcohol. The amount of the at least one aliphatic alcohol in the present oral dosage form will be determined, as above, by the precise rate of opioid release required. It will also depend on whether at least one polyalkylene glycol is present in or absent from the oral dosage form. In the absence of at least one polyalkylene glycol, the oral dosage form preferably contains between 20% and 50% (by wt) of the at least one aliphatic alcohol. When at least one polyalkylene glycol is present in the oral dosage form, then the combined weight of the at least one aliphatic alcohol and the at least one polyalkylene glycol preferably constitutes between 20% and 50% (by wt) of the total dosage.

In one embodiment, the ratio of, e.g., the at least one hydroxyalkyl cellulose or acrylic resin to the at least one aliphatic alcohol/polyalkylene glycol determines, to a considerable extent, the release rate of the opioid from the formulation. A ratio of the at least one hydroxyalkyl cellulose to the at least one aliphatic alcohol/polyalkylene glycol of between 1:2 and 1:4 is preferred, with a ratio of between 1:3 and 1:4 being particularly preferred.

The at least one polyalkylene glycol may be, for example, polypropylene glycol or, which is preferred, polyethylene glycol. The number average molecular weight of the at least one polyalkylene glycol is preferred between 1,000 and 15,000 especially between 1,500 and 12,000.

Another suitable controlled release matrix would comprise an alkylcellulose (especially ethyl cellulose), a C₁₂ to C₃₆ aliphatic alcohol and, optionally, a polyalkylene glycol.

In another preferred embodiment, the matrix includes a pharmaceutically acceptable combination of at least two hydrophobic materials.

In addition to the above ingredients, a controlled release matrix may also contain suitable quantities of other materials, e.g. diluents, lubricants, binders, granulating aids, colorants, flavorants and glidants that are conventional in the pharmaceutical art.

Processes for Preparing Matrix-Based Beads

In order to facilitate the preparation of a solid, controlled release, oral dosage form according to this invention, any method of preparing a matrix formulation known to those skilled in the art may be used. For example incorporation in the matrix may be effected, for example, by (a) forming granules comprising at least one water soluble hydroxyalkyl cellulose and opioid or an opioid salt; (b) mixing the hydroxyalkyl cellulose containing granules with at least one C₁₂-C₃₆ aliphatic alcohol; and (c) optionally, compressing and shaping the granules. Preferably, the granules are formed by wet granulating the hydroxyalkyl cellulose/opioid with water. In a particularly preferred embodiment of this process, the amount of water added during the wet granulation step is preferably between 1.5 and 5 times, especially between 1.75 and 3.5 times, the dry weight of the opioid.

In yet other alternative embodiments, a spheronizing agent, together with the active ingredient can be spheronized to form spheroids. Microcrystalline cellulose is preferred. A suitable microcrystalline cellulose is, for example, the material sold as Avicel PH 101 (Trade Mark, FMC Corporation). In such-embodiments, in addition to the active ingredient and spheronizing agent, the spheroids may also contain a binder. Suitable binders, such as low viscosity, water soluble polymers, will be well known to those skilled in the pharmaceutical art. However, water soluble hydroxy lower alkyl cellulose, such as hydroxypropylcellulose, are preferred. Additionally (or alternatively) the spheroids may contain a water insoluble polymer, especially an acrylic polymer, an acrylic copolymer, such as a methacrylic acid-ethyl acrylate co-polymer, or ethyl cellulose. In such embodiments, the sustained release coating will generally include a hydrophobic material such as (a) a wax, either alone or in admixture with a fatty alcohol; or (b) shellac or zein.

Melt Extrusion Matrix

Sustained release matrices can also be prepared via melt-granulation or melt-extrusion techniques. Generally, melt-granulation techniques involve melting a normally solid hydrophobic material, e.g. a wax, and incorporating a powdered drug therein. To obtain a sustained release dosage form, it may be necessary to incorporate an additional hydrophobic substance, e.g. ethylcellulose or a water-insoluble acrylic polymer, into the molten wax hydrophobic material. Examples of sustained release formulations prepared via melt-granulation techniques are found in U.S. Pat. No. 4,861,598, hereby incorporated by reference in its entirety.

The additional hydrophobic material may comprise one or more water-insoluble wax-like thermoplastic substances possibly mixed with one or more wax-like thermoplastic substances being less hydrophobic than said one or more water-insoluble wax-like substances. In order to achieve constant release, the individual wax-like substances in the formulation should be substantially non-degradable and insoluble in gastrointestinal fluids during the initial release phases. Useful water-insoluble wax-like substances may be those with a water-solubility that is lower than about 1:5,000 (w/w).

In addition to the above ingredients, a sustained release matrix may also contain suitable quantities of other materials, e.g., diluents, lubricants, binders, granulating aids, colorants, flavorants and glidants that are conventional in the pharmaceutical art. The quantities of these additional materials will be sufficient to provide the desired effect to the desired formulation. In addition to the above ingredients, a sustained release matrix incorporating melt-extruded multiparticulates may also contain suitable quantities of other materials, e.g. diluents, lubricants, binders, granulating aids, colorants, flavorants and glidants that are conventional in the pharmaceutical art in amounts up to about 50% by weight of the particulate if desired.

Specific examples of pharmaceutically acceptable carriers and excipients that may be used to formulate oral dosage forms are described in the Handbook of Pharmaceutical Excipients, American Pharmaceutical Association (1986), incorporated by reference herein.

Melt Extrusion Multiparticulates

The preparation of a suitable melt-extruded matrix according to the present invention may, for example, include the steps of blending the opioid analgesic, together with at least one hydrophobic material and preferably the additional hydrophobic material to obtain a homogeneous mixture. The homogeneous mixture is then heated to a temperature sufficient to at least soften the mixture sufficiently to extrude the same. The resulting homogeneous mixture is then extruded to form strands. The extrudate is preferably cooled and cut into multiparticulates by any means known in the art. The strands are cooled and cut into multiparticulates. The multiparticulates are then divided into unit doses. The extrudate preferably has a diameter of from about 0.1 to about 5 mm and provides sustained release of the therapeutically active agent for a time period of from about 8 to about 24 hours.

An optional process for preparing the melt extrusions of the present invention includes directly metering into an extruder a hydrophobic material, a therapeutically active agent, and an optional binder; heating the homogenous mixture; extruding the homogenous mixture to thereby form strands; cooling the strands containing the homogeneous mixture; cutting the strands into particles having a size from about 0.1 mm to about 12 mm; and dividing said particles into unit doses. In this aspect of the invention, a relatively continuous manufacturing procedure is realized.

The diameter of the extruder aperture or exit port can also be adjusted to vary the thickness of the extruded strands. Furthermore, the exit part of the extruder need not be round; it can be oblong, rectangular, etc. The exiting strands can be reduced to particles using a hot wire cutter, guillotine, etc.

The melt extruded multiparticulate system can be, for example, in the form of granules, spheroids or pellets depending upon the extruder exit orifice. For purposes of the present invention, the terms “melt-extruded multiparticulate(s)” and “melt-extruded multiparticulate system(s)” and “melt-extruded particles” shall refer to a plurality of units, preferably within a range of similar size and/or shape and containing one or more active agents and one or more excipients, preferably including a hydrophobic material as described herein. In this regard, the melt-extruded multiparticulates will be of a range of from about 0.1 to about 12 mm in length and have a diameter of from about 0.1 to about 5 mm. In addition, it is to be understood that the melt-extruded multiparticulates can be any geometrical shape within this size range. Alternatively, the extrudate may simply be cut into desired lengths and divided into unit doses of the therapeutically active agent without the need of a spheronization step.

In one preferred embodiment, oral dosage forms are prepared to include an effective amount of melt-extruded multiparticulates within a capsule. For example, a plurality of the melt-extruded multiparticulates may be placed in a gelatin capsule in an amount sufficient to provide an effective sustained release dose when ingested and contacted by gastric fluid.

In another preferred embodiment, a suitable amount of the multiparticulate extrudate is compressed into an oral tablet using conventional tableting equipment using standard techniques. Techniques and compositions for making tablets (compressed and molded), capsules (hard and soft gelatin) and pills are also described in Remington's Pharmaceutical Sciences, (Arthur Osol, editor), 1553-1593 (1980), incorporated by reference herein.

In yet another preferred embodiment, the extrudate can be shaped into tablets as set forth in U.S. Pat. No. 4,957,681 (Klimesch, et. al.), described in additional detail above and hereby incorporated by reference.

Optionally, the sustained release melt-extruded multiparticulate systems or tablets can be coated, or the gelatin capsule can be further coated, with a sustained release coating such as the sustained release coatings described above. Such coatings preferably include a sufficient amount of hydrophobic material to obtain a weight gain level from about 2 to about 30 percent, although the overcoat may be greater depending upon the physical properties of the particular opioid analgesic compound utilized and the desired release rate, among other things.

The melt-extruded unit dosage forms of the present invention may further include combinations of melt-extruded multiparticulates containing one or more of the therapeutically active agents disclosed above before being encapsulated. Furthermore, the unit dosage forms can also include an amount of an immediate release therapeutically active agent for prompt therapeutic effect. The immediate release therapeutically active agent may be incorporated, e.g., as separate pellets within a gelatin capsule, or may be coated on the surface of the multiparticulates after preparation of the dosage forms (e.g., controlled release coating or matrix-based). The unit dosage forms of the present invention may also contain a combination of controlled release beads and matrix multiparticulates to achieve a desired effect.

The sustained release formulations of the present invention preferably slowly release the therapeutically active agent, e.g., when ingested and exposed to gastric fluids, and then to intestinal fluids. The sustained release profile of the melt-extruded formulations of the invention can be altered, for example, by varying the amount of retardant, i.e., hydrophobic material, by varying the amount of plasticizer relative to hydrophobic material, by the inclusion of additional ingredients or excipients, by altering the method of manufacture, etc.

In other embodiments of the invention, the melt extruded material is prepared without the inclusion of the therapeutically active agent, which is added thereafter to the extrudate. Such formulations typically will have the therapeutically active agent blended together with the extruded matrix material, and then the mixture would be tableted in order to provide a slow release formulation. Such formulations may be advantageous, for example, when the therapeutically active agent included in the formulation is sensitive to temperatures needed for softening the hydrophobic material and/or the retardant material.

### DETAILED DESCRIPTION OF PREFERRED EMBODIMENTS

The following examples illustrate various aspects of the present invention. They are not to be construed to limit the claims in any manner whatsoever.

A direct comparison of the competitive opioid receptor binding properties of 6-beta-naltrexol or other neutral receptor binding agents following its co-administration with various opioid agonists has not been undertaken previous to the present invention, to the knowledge of the inventor. However, such run-up dosing regimens are easily implemented by those skilled in the art in light of the present invention.

The scope of the invention is not intended to be limited by the ratios of neutral receptor binding agent to opioid agonist analgesic within a pharmaceutical composition recited herein because optimal such ratios can only be determined by experimentation that in light of the present invention is routine, typical and expected of such an invention, and which is readily carried out by one skilled in the art. Further, in the experiments carried out by Kaiko and Colucci in '494, the interactions of naltrexone with 6-beta-naltrexol in vivo are not predicted by their work in that the serum half-life of 6-beta-naltrexol is longer than the time period used to evaluate area under the curve (AUC) in relation to naltrexone (see FIG. 11, e.g., which record AUC at 6 hours for varying doses of naltrexone). Further, it is difficult to extrapolate the effect of a plasma concentration of 6-beta-naltrexol alone when compared to a plasma containing concentrations of both naltrexone and 6-beta-naltrexone. For instance, how much of the competitive binding of opioid receptors, irrespective of efficacy or effect on receptor intrinsic activity, is due to the naltrexone or due to 6-beta-naltrexol? This question can be answered, however, as taught herein, by examining the various effects (data points as previously described) associated with given concentrations of naltrexone and the metabolite 6-beta-naltrexol, respectively (which are both easily measured, and which were measured by Kaiko and Colucci, though not directly addressed in '494), and the same various effects associated with the same given concentration(s) of 6-beta-naltrexol (in absence of naltrexone), and then by subtracting out the 6-beta-naltrexol effects from both samples. One can then estimate the effects due to naltrexone alone and 6-beta-naltrexol alone. Kaiko and Colucci make no mention of such efforts. Of course, that those skilled in the art as Kaiko and Colucci are, have not described combining a neutral receptor binding agent with an opioid agonist analgesic, despite all the work in this general area done by such skilled artisans, speaks strongly to the fact the present invention is neither obvious, nor taught by the prior art. It is clear that there is utility of the present invention, and it is still further apparent that the present invention has a great commercial marketplace. That Sadee and Wang also have not taught or contemplated the present invention is further evidence of its non-obviousness.

'494 also doesn't take into consideration dosing over a given time period. It addresses taking a larger amount than prescribed (2-3 times the prescribed amount) of an opioid analgesic preparation as a single bolus, but doesn't address, e.g., taking the prescribed dose more often than prescribed, or taking the prescribed or excessive dose over a period of several days. This is important because the half-life of hydrocodone is approximately 3.8 hours, while that of naltrexone is approximately 4 hours, and that of 6-beta-naltrexol 13 hours (Physicians' Desk Reference, 54^{th} ed, 2000). Though the half-life of hydrocodone matches up fairly well with naltrexone, over time 6-beta-naltrexol will tend to accumulate relative to hydrocodone (when administered together), such that maximum steady-state effects of 6-beta-naltrexol will not be seen for several days. This is the case whether 6-beta-naltrexol is administered in the absence or presence of naltrexone. Kaiko and Colucci appear to address only steady state effects relating to hydrocodone and naltrexone, ignoring the component effect inherent in the present invention, i.e., the effect of “just” 6-beta-naltrexol. This is not a trivial matter, because 6-beta-naltrexol concentrations arising secondarily from metabolism of the parent naltrexone may vary greatly and are not predictable enough for a consistent effect of a pharmaceutical composition (see, for examples, “Serum time course of naltrexone and 6beta-naltrexol levels during long term treatment in drug addicts” by Ferrari, et al. in Drug and Alcohol Dependence, Vol. 52, pp. 211-220, 1998, and “Kinetics and inhibition of the formation of 6beta-naltrexol from naltrexone in human liver cytosol” by Porter, et al. in British Journal of Clinical Pharmacology, Vol. 50, pp. 465-472, 2000). Further, Kaiko and Colucci do not distinguish “aversive effects” among and between opioid naïve humans and opioid dependent humans. Thus, it appears that Kaiko and Colucci in no way anticipate the present invention.

The present invention is also not intended to be limited by theoretical mechanism, but rather to comprise the spirit and scope of the specifications herein including its claims and any subsequent allowed claims.

### EXAMPLE 26

Ideally, 6-beta-naltrexone is administered in such a fashion such that steady state concentration of it is reached in about four and a half half-lives, or in about two and a half days. Thus, the goal is to have a steady state concentration of 6-beta-naltrexol at two and a half days that is below threshold for competing with co-administered opioid agonist analgesic (e.g., hydrocodone, oxycodone) to any clinically significant extent which by that time has also reached its own steady-state concentration. Therefore, one must determine at what concentration in a given individual (an opioid naïve human, or an opioid-dependent human) 6beta-naltrexol will not noticably adversely alter effective analgesia due to the opioid agonist analgesic. Ideally, this threshold is one that is overcome by taking either more than the prescribed dose of analgesic after two and a half days or taking the analgesic more often than prescribed after two and a half days. That concentration of 6-beta-naltrexol (or other neutral receptor binding agent) may or may not be independent of the concomitant serum concentration of opioid agonist analgesic. Whether or not it is independent depends upon the relative potencies, or affinities for the mu-opioid receptor, of the 6-beta-naltrexol and opioid agonist. We know that in the opioid dependent model, the efficacy of 6-beta-naltrexol is a net zero, neither increasing or decreasing the intrinsic activity of the opioid receptor, and that in all cases the efficacy of the opioid agonist analgesic is significant. Therefore, the determining factor for whether or not the sub-threshold steady-state concentration of 6-beta-naltrexol is dependent upon the concentration of the opioid agonist is the relative potencies of the neutral receptor binding agent (e.g., 6-beta-naltrexol) and the opioid agonist analgesic (e.g., hydrocodone, oxycodone). Potencies of various opioid analgesics are known to those skilled in the art insofar as much some practitioners of the art hold such information as trade secrets. In vivo and in vitro potencies of 6-beta-naltrexol are described by Porter et al. in “In vivo and in vitro potency studies of 6beta-naltrexol, the major human metabolite of naltrexone,” (Addiction Biology, Vol. 7, No. 2, pp. 219-25, April 2002—the abstract of which is incorporated by way of reference). The ideal situation is when the relative potencies allow for a sub-threshold steady-state concentration of neutral receptor binding agent that tends to be independent of the opioid agonist analgesic concentration. In such cases, then a dose of 6-beta-naltrexol can be calculated from experimentation, in light of the present invention, that is constant (“D_{6BN}”). D_{6BN} is then formulated with differing doses of opioid analgesic such as to create a “library” of pharmaceutical compositions, each comprising a specific dose of opioid agonist analgesic, D_{6BN}, and a pharmaceutical carrier thereof For example, oxycodone is commercially available from several U.S. pharmaceutical concerns in a number of dosage forms of varying dose, e.g., 10 mg, 20 mg, 40 mg and 80 mg (from Purdue Pharma, LLP, Stamford, Conn.). The present invention then, teaches various pharmaceutical compositions containing, e.g., 10 mg oxycodone and D_{6BN-oxy}, 20 mg oxycodone and D_{6BN-oxy}, 40 mg oxycodone and D_{6BN-oxy}, and 80 my oxycodone and D_{6BN-oxy}, depending upon, of course, the relative potency of 6-beta-naltrexol to oxycodone. Alternatively, the present invention teaches in a like manner, various pharmaceutical compositions containing, e.g., 5 mg hydrocodone and D_{6BN-hyd}, 70.5 mg hydrocodone and D_{6BN-hyd}, and 10 mg hydrocodone and D_{6BN-hyd} (where “D_{6BN-hyd}” is the dose of 6-beta-naltrexol resulting in steady-state blood concentration of 6-beta-naltrexol that is immediately sub-threshold to diminishing effective analgesia of therapeutic doses of oxycodone, and “D_{6BN-hyd}” is the dose of 6-beta-naltrexol resulting in steady-state blood concentration of 6-beta-naltrexol that is immediately sub-threshold to diminishing effective analgesia of therapeutic doses of hydrocodone).

This is accomplished in the following way.

An easily comparable number of groups of humans, e.g., A, B, C . . . N, matching in general demographic characteristics (e.g., sex, age, etc.), of sufficient number to yield meaningful results, are grouped as follows:

| | |
|---|---|
| Group A: | Opioid naïve humans receiving 15 mg hydrocodone and a |
| | hypothetical dose of 6-beta-naltrexol, D_{6BN-hyd}, e.g., 0.5 mg |
| | of 6-beta-naltrexol every 6 hours |
| Group B: | Opioid naïve humans receiving 15 mg hydrocodone and a |
| | hypothetical dose of 6-beta-naltrexol, D_{6BN-hyd}, e.g., 1.0 mg |
| | of 6-beta-naltrexol every 6 hours |
| Group C: | Opioid naïve humans receiving 15 mg hydrocodone and a |
| | hypothetical dose of 6-beta-naltrexol, D_{6BN-hyd}, e.g, 2.0 mg |
| | of 6-beta-naltrexol every 6 hours |
| Group D: | Opioid naïve humans receiving 15 mg hydrocodone and a |
| | hypothetical dose of 6-beta-naltrexol, D_{6BN-hyd}, e.g., 4.0 mg |
| | of 6-beta-naltrexol every 6 hours |
| Group E: | Opioid naïve humans receiving 15 mg hydrocodone and a |
| | hypothetical dose of 6-beta-naltrexol, D_{6BN-hyd}, e.g. 8.0 mg |
| | of 6-beta-naltrexol every 6 hours |
| Group F: | Opioid naïve humans receiving 15 mg hydrocodone and a |
| | hypothetical dose of 6-beta-naltrexol, D_{6BN-hyd}, e.g., 16 mg |
| | of 6-beta-naltrexol every 6 hours |
| Group G: | Opioid dependent humans receiving 15 mg hydrocodone and a |
| | hypothetical dose of 6-beta-naltrexol, D_{6BN-hyd}, e.g., 0.5 mg |
| | of 6-beta-naltrexol every 6 hours |
| Group H: | Opioid dependent humans receiving 15 mg hydrocodone and a |
| | hypothetical dose of 6-beta-naltrexol, D_{6BN-hyd}, e.g. 1.0 mg |
| | of 6-beta-naltrexol every 6 hours |
| Group I: | Opioid dependent humans receiving 15 mg hydrocodone and a |
| | hypothetical dose of 6-beta-naltrexol, D_{6BN-hyd}, e.g., 2.0 mg |
| | of 6-beta-naltrexol every 6 hours |
| Group J: | Opioid dependent humans receiving 15 mg hydrocodone and a |
| | hypothetical dose of 6-beta-naltrexol, D_{6BN-hyd}, e.g., 4.0 mg |
| | of 6-beta-naltrexol every 6 hours |
| Group K: | Opioid dependent humans receiving 15 mg hydrocodone and a |
| | hypothetical dose of 6-beta-naltrexol, D_{6BN-hyd}, e.g., 8.0 mg |
| | of 6-beta-naltrexol every 6 hours |
| Group L: | Opioid dependent humans receiving 15 mg hydrocodone and a |
| | hypothetical dose of 6-beta-naltrexol, D_{6BN-hyd}, e.g, 16 mg |
| | of 6-beta-naltrexol every 6 hours |
| Group M: | Opioid naïve humans receiving 15 mg hydrocodone and no 6- |
| | beta-naltrexol every six hours |
| Group N: | Opioid dependent humans receiving 15 mg hydrocodone and |
| | no 6-beta-naltrexol every 6 hours |

Each group is tested over approximately three days or more, measuring the following parameters at reasonable intervals (e.g., every 6 hours): i) serum hydrocodone concentration, ii) serum 6-beta-naltrexol concentration, iii) ARCI scores, iv) POMS scores, v) visual analog scores (“VAS”) as described by Kaiko and Colucci, vi) pupil size as in routine pupilometry or as described by Kaiko and Colucci, vii) the electrical current presented to the skin that is associated with a particular score on a scale measuring discomfort due to administration of the electrical current to the skin of the human subject, viii) scales measuring discomfort due to administration of the electrical current to the skin of the human subject. A ninth parameter that may be measured is electroencephalographic (“EEG”) activity from the scalp of the human subject. A tenth parameter that may be measured is respiratory rate. An eleventh parameter that may be measured expired end-tidal carbon dioxide, such as by routine capnography. A twelfth parameter that may be measured is blood pressure by non-invasive sphygmomanometry. A thirteenth parameter that may be measured is pulse rate or heart rate by manual palpation, electrocardiography (“ECG”) or pulse oximetry. A Modified Specific Drug Effect Questionnaire (“MSDEQ”) may also be employed as described in '494.

ARCI, POMS and VAS correlate with “liking” of the pharmaceutical preparation administration and may be used to estimate euphoric effects. Pupillary response is associated with mu opoiod activity and pupillary miosis correlates with opioid agonist activity and pupillary dilatation correlates with opioid withdrawal or possibly inverse agonist effects. There are EEG correlates for euphoria (e.g., increased alpha activity as described by Lukas, et al.—“EEG alpha activity increases during transient episodes of ethanol-induced euphoria” in Pharmacology, Biochemistry and Behavior, Vol. 25, No. 4, pp. 889-95, October 1986, and by Lukas, et al.—“Electroencephalographic correlates of marihuana-induced euphoria” in Drug and Alcohol Dependence, Vol. 37, No. 2, pp. 131-40, February 1995) and pain (see Chang, et al., “Differential cerebral responses to aversive auditory arousal versus muscle pain: specific EEG patterns are associated with human pain processing” in Experimental Brain Research, Vol. 147, No. 3, pp. 387-93, December 2002, and Chang, et al., “Psychophysical and EEG responses to repeated experimental muscle pain in humans: pain activity encodes EEG activity” in Brain Research Bulletin, Vol. 59, No. 6, pp. 533-43, February 2003). There is an inverse relationship between respiratory rate and mu opioid agonist activity. There is a proportional or direct relationship between end-tidal carbon dioxide amount and opioid agonist activity. Opioid agonist analgesics tend to decrease resting heart rate and blood pressure, while opioid withdrawal (as associated with inverse opioid agonist activity in opioid dependent individuals) tends to increase resting heart rate and blood pressure.

The 14 pharmaceutical aliquot preparations containing 6-beta-naltrexol are made up as follows:

Lorcet 10/650 tablets (Forest Laboratories, Inc., St. Louis, Mo.), each containing 10 mg hydrocodone bitartrate and 650 mg acetaminophen, used. Tablets are easily cut in half with the razor blade of a commercially available “pill splitter.” 15 tablets are ground up into a powder by mortar and pestal. To it is added 6-beta-naltrexol powder in a pre-measured amount, obtained from Mallinckrodt Chemical of St. Louis, Mo. To make a 15 mg hydrocodone bitartrate/975 mg acetaminophen/0.5 mg 6-beta-naltrexol preparation, 5 mg of the 6-beta-naltrexol powder is mixed thoroughly with the powder from 10 Lorcet 10/650 tablets. The 5 mg is measured by an A&D brand GX-200 top loading balance (purchased from Spectrum Chemical & Laboratory Products, Gardena, California, catalog number 440-68329). After thorough mixing, the mixed powder from ten Lorcet 10/650 tablets and 5 mg 6-beta-naltrexol powder is measured on the GX-200 balance. One tenth of the measured powder is separated and put into a gelatin-based enterally dissolvable capsule of appropriate size (as can be purchased from a variety of pharmacy compounding supply companies in the United States). To form the other preparations, one simply doubles the amount of 6-beta-naltrexol in each subsequent aliquot until a preparation containing 15 mg hydrocodone bitartrate/975 mg acetaminophen/16 mg 6-beta-naltrexol is arrive at. Ideally, one would add an amount of inert ingredient as needed such that all the capsules have approximately equal mass as measured on the balance, though there is less of a need for this if the experimental protocol calls for placing each capsule in the mouth of the human subject by the experimenter so as negate the likelihood that human participant will realize any appreciable difference in weight of the capsules, which they will be administered in set time intervals, e.g., every three to six hours or so, or having a time interval approximately equal to the serum half-life of the shortest acting active drug component of the combination pharmaceutical composition.

The subjects given no 6-beta-naltrexol may simply be administered one and one half tablets of Lorcet 10/650 ground by motor and pestal into a powder and placed into a gelatin capsule.

These methods of compounding are “best” only in terms of simplicity for the sake of explanation, are not meant to be interpreted as a best mode of manufacture. Superior methods of manufacture, in light of the present invention, would become apparent to one so skilled in the art of pharmaceutical manufacture.

By collecting and analyzing data in the usual fashion for the above-referenced parameters over time, an approximate of the immediate sub-threshold concentration of 6-beta-naltrexone at steady state, D_{6BN-hyd}, will become readily apparent to one skilled in the art.

### EXAMPLE 27

The invention described above except that instead of including a neutral receptor binding agent the invention includes a relatively low efficacy opioid agonist analgesic or a partial mu-opioid agonist. This partial mu-opioid agonist may be nalbuphine.

### EXAMPLE 28

The invention described herein where the opioid agonist analgesic providing for effective analgesia is noroxycodone. This is a significant improvement over prior art technology in that the advantages of noroxycodone as an analgesic over oxycodone as an analgesic have not been appreciated by those skilled in the art of pharmaceutical manufacture or marketing. Analgesic effects due to the parent oxycodone are primarily kappa opioid receptor mediated (see Ross and Smith, “The intrinsic antinociceptive effects of oxycodone appear to be kappa-opioid receptor mediated” in Pain, Vol. 73, No. 2, pp. 151-7, November 1997), while the analgesic effects due to noroxycodone are relatively less kappa opioid receptor mediated, even though noroxycodone may have a relatively lower efficacy than its parent compound. Thus, when oxycodone is administered, there undergoes a transformation, not appreciated until the light of the present invention, whereby there is a transition from relatively more kappa dominated opioid effects to relatively less kappa dominated opioid effect, as the metabolite noroxycodone increases its relative concentration to oxycodone. That this was not appreciated by those very practitioners responsible for oxycodone is evidenced by Kaiko, et al. in “Pharmacokinetic-pharmacodynamic relationships of controlled-release oxycodone” (Clinical Pharmacology and Therapeutics, Vol. 59, No. 1, pp. 52-61, January 1996) where the authors conclude “these results support oxycodone, and not oxymorphone [another metabolite of oxycodone], as being responsible for pharmacodynamic and analgesic effects.” When oxycodone is initially administered to opioid naïve (oxycodone naïve) humans, there is an increased likelihood of dysphoria and/or nausea/vomiting initially. The present author attributes these initial ill effects to the relative domination of kappa effects, which the author has previously described in U.S. Pat. Nos. 5,783,583 and 6,103,258. As time goes by, the patient “gets used to” being administered oxycodone and the dysphoria, nausea and/or vomiting tend to subside. This, the present invention claims, is due to a relative shift away from kappa effects as oxycodone is converted to noroxycodone.

### EXAMPLE 29

The invention described herein where the relatively low efficacy opioid agonist analgesic or partial mu-opioid agonist or neutral binding agent is “CTAP” (the D-Phe-Cys-Tyr-D-Trp-Arg-Thr-Pen-Thr-NH2 cyclic, penicillamine-containing octapeptide described by Abbruscato, et al., “Blood-Brain Barrier Permability and Bioavailability of a Highly Potent and mu-Selective Opioid Receptor Antagonist, CTAP: Comparison with Morphine” in The Journal of Pharmacology and Experimental Therapeutics, Vol. 280, No. 1, pp. 402-409, 1997).

### EXAMPLE 30

The invention described herein where the relatively low efficacy opioid agonist analgesic or partial mu-opioid agonist or neutral binding agent is xorphanol, described by Gharagozlou, et al (Ibid).

While the invention has been described and illustrated with reference to certain preferred embodiments thereof, those skilled in the art will appreciate that obvious modifications can be made herein without departing from the spirit and scope of the invention. Such variations are contemplated to be within the scope of the appended claims.

I claim by letters patent:

1.) A combination pharmaceutical preparation comprising:
an opioid agonist analgesic;
a neutral receptor binding agent;
and, an acceptable pharmaceutical carrier thereof.

2.) A combination pharmaceutical preparation comprising:
an opioid agonist analgesic;
a partial mu-opioid agonist;
and, an acceptable pharmaceutical carrier thereof.

3.) The invention of claim 1 where the neutral receptor binding agent is any one from the group consisting of 6-alpha-naltrexol, 6-beta-naltrexol, 6-alpha-naloxol, 6-beta-naloxol and 6-beta-naltrexamine.

4.) The invention of claim 2 where said partial mu-opioid agonist is nalbuphine.

5.) An opioid agonist analgesic composition comprising noroxycodone and a suitable pharmacological carrier thereof.

6.) A combination pharmaceutical preparation comprising:
an opioid agonist analgesic;
CTAP;
and, an acceptable pharmaceutical carrier thereof.

7.) A combination pharmaceutical preparation comprising:
an opioid agonist analgesic;
xorphanol;
and, an acceptable pharmaceutical carrier thereof.

8.) The invention of claim 1 where said opioid agonist analgesic is any one from the group consisting of alfentanil, allylprodine, alphaprodine, anileridine, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, fentanyl, heroin, hydrocodone, hydromorphone, hydroxypethidine, isomethadone, ketobemidone, levorphanol, levophenacylmorphan, lofentanil, meperidine, meptazinol, metazocine, methadone, metopon, morphine, myrophine, narceine, nicomorphine, norlevorphanol, normethadone, noroxycodone, nalorphine, nalbuphene, normorphine, norpipanone, opium, oxycodone, oxymorphone, papaveretum, pentazocine, phenadoxone, phenomorphan, phenazocine, phenoperidine, piminodine, piritramide, propheptazine, promedol, properidine, propoxyphene, sufentanil, tilidine and tramadol.

9.) The invention of claim 2 where said opioid agonist analgesic is any one from the group consisting of alfentanil, allylprodine, alphaprodine, anileridine, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, fentanyl, heroin, hydrocodone, hydromorphone, hydroxypethidine, isomethadone, ketobemidone, levorphanol, levophenacylmorphan, lofentanil, meperidine, meptazinol, metazocine, methadone, metopon, morphine, myrophine, narceine, nicomorphine, norlevorphanol, normethadone, noroxycodone, nalorphine, nalbuphene, normorphine, norpipanone, opium, oxycodone, oxymorphone, papaveretum, pentazocine, phenadoxone, phenomorphan, phenazocine, phenoperidine, piminodine, piritramide, propheptazine, promedol, properidine, propoxyphene, sufentanil, tilidine and tramadol.

10.) The invention of claim 3 where said opioid agonist analgesic is any one from the group consisting of alfentanil, allylprodine, alphaprodine, anileridine, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, fentanyl, heroin, hydrocodone, hydromorphone, hydroxypethidine, isomethadone, ketobemidone, levorphanol, levophenacylmorphan, lofentanil, meperidine, meptazinol, metazocine, methadone, metopon, morphine, myrophine, narceine, nicomorphine, norlevorphanol, normethadone, noroxycodone, nalorphine, nalbuphene, normorphine, norpipanone, opium, oxycodone, oxymorphone, papaveretum, pentazocine, phenadoxone, phenomorphan, phenazocine, phenoperidine, piminodine, piritramide, propheptazine, promedol, properidine, propoxyphene, sufentanil, tilidine and tramadol.

11.) The invention of claim 4 where said opioid agonist analgesic is any one from the group consisting of alfentanil, allylprodine, alphaprodine, anileridine, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethytmethylthiambutene, ethylmorphine, etonitazene, fentanyl, heroin, hydrocodone, hydromorphone, hydroxypethidine, isomethadone, ketobemidone, levorphanol, levophenacylmorphan, lofentanil, meperidine, meptazinol, metazocine, methadone, metopon, morphine, myrophine, narceine, nicomorphine, norlevorphanol, normethadone, noroxycodone, nalorphine, nalbuphene, normorphine, norpipanone, opium, oxycodone, oxymorphone, papaveretum, pentazocine, phenadoxone, phenomorphan, phenazocine, phenoperidine, piminodine, piritramide, propheptazine, promedol, properidine, propoxyphene, sufentanil, tilidine and tramadol.

12.) The invention of claim 6 where said opioid agonist analgesic is any one from the group consisting of alfentanil, allylprodine, alphaprodine, anileridine, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, fentanyl, heroin, hydrocodone, hydromorphone, hydroxypethidine, isomethadone, ketobemidone, levorphanol, levophenacylmorphan, lofentanil, meperidine, meptazinol, metazocine, methadone, metopon, morphine, myrophine, narceine, nicomorphine, norlevorphanol, normethadone, noroxycodone, nalorphine, nalbuphene, normorphine, norpipanone, opium, oxycodone, oxymorphone, papaveretum, pentazocine, phenadoxone, phenomorphan, phenazocine, phenoperidine, piminodine, piritramide, propheptazine, promedol, properidine, propoxyphene, sufentanil, tilidine and tramadol.

13.) The invention of claim 7 where said opioid agonist analgesic is any one from the group consisting of alfentanil, allylprodine, alphaprodine, anileridine, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, fentanyl, heroin, hydrocodone, hydromorphone, hydroxypethidine, isomethadone, ketobemidone, levorphanol, levophenacylmorphan, lofentanil, meperidine, meptazinol, metazocine, methadone, metopon, morphine, myrophine, narceine, nicomorphine, norlevorphanol, normethadone, noroxycodone, nalorphine, nalbuphene, normorphine, norpipanone, opium, oxycodone, oxymorphone, papaveretum, pentazocine, phenadoxone, phenomorphan, phenazocine, phenoperidine, piminodine, piritramide, propheptazine, promedol, properidine, propoxyphene, sufentanil, tilidine and tramadol.
